(19) United States
(12) Patent Application Publication
(10) Pub. No.: US 2006/0235457 A1
(43) Pub. Date: Oct. 19, 2006

(54) Instruments having a rigidizable external working channel

(76) Inventor: Amir Belson, Sunnyvale, CA (US)

Correspondence Address: WILSON SONSINI GOODRICH & ROSATI, 650 PAGE MILL ROAD, PALO ALTO, CA, 94304-1050, US

(21) Appl. No.: 11/107,503

(22) Filed: Apr. 15, 2005

(51) Int. Cl. A61M 29/00

(52) U.S. Cl. 606/191

(57) ABSTRACT

Embodiments of the present invention are directed towards instruments for investigation, screening, diagnosis, analysis or therapy and, more particularly, towards embodiments of one or more external rigidizable working channels along the instrument. In one embodiment, there is an apparatus that includes an instrument having an elongate body; a working channel connected externally to the elongate body and extending from a proximal position on the elongate body to a distal position on the elongate body, the working channel having a stowed configuration and a deployed configuration; and a plurality of rigidizable elements disposed within the working channel to selectively hold the shape of the working channel when the working channel is in the deployed configuration. Other embodiments include an method of providing a working channel within the body that are positioning an instrument within the body; providing along the instrument an external working channel having a lumen, the lumen extending along the working channel and outside of the instrument; positioning the external working channel into a deployed configuration; and holding the shape of the external working channel in the deployed configuration.

### TECHNICAL FIELD

Embodiments of the present invention are directed towards instruments for investigation, screening, diagnosis, analysis or therapy and, more particularly, towards embodiments of one or more external working channels along the instrument that may be locked into a position or rigidized into a position.

### BACKGROUND OF THE INVENTION

The use of customized instruments or scopes has found widespread use in both medical and non-medical industrial fields. In non-medical industrial applications, customized instruments may be used to investigate the internal condition of components, such as the internal condition of an engine or air intake, the condition of piping system or other conduits and other investigatory or investigatory/repair procedures. Another industrial application is the use of instruments for remote visual inspection and/or repair of difficult to reach areas including those areas in an environment potentially harmful to humans.

In medical applications, the use of intrabody medical instruments, such as endoscopes, catheters, and the like, for screening, diagnostic and therapeutic indications is rapidly expanding. To improve performance, such equipment has been optimized to best accomplish their intended purposes. As examples, endoscopes have been optimized and refined so as to provide upper endoscopes for the examination of the esophagus, stomach, and duodenum, colonoscopes for examining the colon, angioscopes for examining blood vessels, bronchoscopes for examining bronchi, laparoscopes for examining the peritoneal cavity, arthroscopes for examining joints and joint spaces, nasopharygoscopes for examining the nasal passage and pharynx, toracoscopes for examination of the thorax and intubation scopes for examination of a person's airway.

In medical applications, for example, conventional intrabody instruments have an insertion tube connected at its proximal end to a handle or control body. The insertion tube is adapted to be inserted into a patient's body cavity to perform a selected therapeutic or diagnostic procedure. The insertion tube may also contain an imaging system having optical fibers or the like extending along the length of the insertion tube and terminating at a viewing window and/or imaging system or CCD/CMOS system and may provide access for irrigation, suction, grasping or other functions. The insertion tube is also sized to accommodate one or more internal working channels that extend along the insertion tube. The working channels are adapted to receive conventional endoscopic accessories therethrough. Because the working channel is inside the insertion tube or instrument body, the maximum working channel size is limited by the size of the instrument and the space required by the other endoscope elements or conversely, the instrument size must be increased if a larger diameter working channel is to be provided.

While smaller, more compact instruments are generally desirable, smaller conventional instruments would lead to a corresponding decrease in the size of the available working channel. There is a need therefore for smaller, more compact instruments that remain capable of providing appropriately sized working channels.

### SUMMARY OF THE INVENTION

In one embodiment, there is provided an apparatus including an instrument having an elongate body; a working channel connected externally to the elongate body and extending from a proximal position on the elongate body to a distal position on the elongate body, the working channel having a stowed configuration and a deployed configuration; and a plurality of rigidizable elements disposed within the working channel to selectively hold the shape of the working channel when the working channel is in the deployed configuration. In another aspect, the working channel may be detached from the instrument. In another aspect, the rigidizable elements hold the shape of the working channel using mechanical force, using a shape memory alloy element or using an electroactive polymer element. In another aspect, the plurality of rigidizable elements disposed within the working channel comprises nested rigidizable elements. In one aspect, the instrument is adapted to provide more than one working channel, or each working channel of the more than one working channel may be independently released from the instrument.

In another embodiment, there is provided a method of providing a working channel within the body including positioning an instrument within the body; providing along the instrument an external working channel having a lumen, the lumen extending along the working channel and outside of the instrument; positioning the external working channel into a deployed configuration; and holding the shape of the external working channel in the deployed configuration. In another embodiment, the external working channel may be released from the instrument so that the instrument may move axially with respect to the deployed external working channel. In another alternative, the external working channel remains in the deployed configuration using mechanical force produced by rigidizable elements within the external working channel. In another aspect, the working channel is used to perform a screening, therapy or diagnostic procedure within the body. In another aspect, there is provided a method of adjusting the position of the instrument within the body; providing along the instrument another external working channel having a lumen, the lumen extending along the working channel and outside of the instrument; positioning the another external working channel into a deployed configuration; and holding the shape of the another external working channel in the deployed configuration. In another aspect, the working channel, the another working channel and the instrument are used to perform a screening, therapy or diagnostic procedure within the body.

In still another alternative embodiment, there is provided a method of providing access within the body by positioning an instrument within the body; providing along the instrument a first external working channel having a lumen, the lumen extending along the working channel and outside of the instrument; positioning the first external working channel into a deployed configuration in a first position within the body; holding the shape of the first external working channel in the deployed configuration using rigidizable elements within the first external working channel; moving the instrument to a second position within the body; providing along the instrument a second external working channel having a lumen, the lumen extending along the working channel and outside of the instrument; positioning the second external working channel into a deployed configuration in the second position within the body; and holding the shape of the second external working channel in the deployed configuration using rigidizable elements within the second external working channel. In another aspect, there is provided performing a screening, a therapy or a diagnostic procedure within the body at either the first position or the second position. In another aspect, the first position and the second position are adjacent the heart. In another aspect, the first position and the second position are within the gut. In still another aspect, the screening, therapy or diagnostic procedure within the body relates to the heart. In still another aspect the screening, therapy or diagnostic procedure within the body relates to the gut.

### BRIEF DESCRIPTION OF THE DRAWINGS

FIG. 1A is a perspective view of an embodiment of an instrument with an expandable working channel in a stowed configuration.

FIG. 1B is a perspective view of the instrument of FIG. 1A with the expandable working channel in a deployed configuration.

FIG. 2A is a perspective view of another embodiment of an instrument with another expandable working channel embodiment in a stowed configuration.

FIG. 2B is a perspective view of the instrument of FIG. 2A with the expandable working channel in a deployed configuration.

FIG. 2C is an embodiment of an instrument having an expandable working channel with a representation of a control system.

FIGS. 2D and 2E are section views of a conventional interior working channel (FIG. 2D) and an embodiment of an expandable working channel of the invention (FIG. 2E).

FIG. 2F is an alternative embodiment of the instrument of FIGS. 2A and 2B with a non-solid working channel.

FIGS. 2G, 2H and 21 illustrate views of an external working channel embodiment having a quick release mechanism.

FIGS. 3A-3C illustrate alternative working channel to instrument relationships.

FIGS. 3D-3H illustrate an external working channel embodiment attached to an instrument.

FIGS. 4A and 4B illustrate different working channel internal lumen shape embodiments.

FIGS. 5A-5C illustrate one embodiment of an instrument with two external working channels stowed (FIG. 5A), with one channel deployed (FIG. 5B) and both channels deployed (FIG. 5C).

FIGS. 5D and 5E illustrate another embodiment of an instrument with two external working channels stowed (FIG. 5D) and deployed (FIG. 5E).

FIG. 6A-6B illustrate an embodiment of an instrument with multiple working channels in the stowed (FIG. 6A) and deployed (FIG. 6B) configurations.

FIG. 7-7C illustrate an embodiment of an instrument with multiple working channels in the stowed (FIG. 7) and various deployed configurations.

FIG. 8A-8D illustrate an embodiment of an instrument with multiple working channels in the stowed (FIG. 8A) and various deployed configurations.

FIG. 9-8D illustrate an embodiment of an instrument with multiple working channels in the stowed (FIG. 9) and various deployed configurations.

FIGS. 10-11A illustrate an embodiment of an instrument with an embodiment of a semi-tube working channel in the stowed (FIGS. 10, 10A) and deployed (FIGS. 11, 11A) configurations.

FIGS. 12-13A illustrate an embodiment of an instrument with an embodiment of a semi-tube working channel having an internally expandable lumen in the stowed (FIGS. 12, 12A) and deployed (FIGS. 13, 13A) configurations.

FIGS. 14A-14C illustrate several views of a device advancing distally along an embodiment of a deformable external working channel on an instrument.

FIG. 14D illustrates an external working channel with semi-rigid sections.

FIGS. 15A-15B illustrate cross section end views an embodiment of an external working channel that is larger than the instrument when in the deployed configuration (FIG. 15B).

FIGS. 16-16E illustrate alternative guides and delivery techniques for external working channels.

FIGS. 17A and 17B illustrate the use of a reel to advance an external working channel.

FIG. 18 illustrates the use of a lead screw to advance an external working channel.

FIGS. 19 and 20 illustrate alternative roller based external working channel delivery mechanisms.

FIG. 21 illustrates an instrument having a plurality of guides to receive multiple external working channels.

FIGS. 22A and 22B illustrate a detachable and separately controllable external working channel.

FIG. 23 illustrates an inspection device embodiment.

FIGS. 24-26 illustrate alternative working channel sidewall configurations.

FIGS. 27A-27D illustrate a technique to use the working channel of a conventional instrument to deliver an external working channel embodiment.

FIGS. 27E and 27F illustrate a steerable external working channel embodiment.

FIGS. 28A through 28F illustrate a rigidizable working channel in use around the heart.

FIGS. 29A-29D illustrate the delivery and use of multiple rigidizable working channels.

FIG. 30 illustrates an embodiment of an instrument adapted to deliver multiple external working channels.

FIGS. 31-39C illustrate alternative aspects and further details of the rigidizable elements that may be used in conjunction with a working channel.

FIGS. 40-41B illustrate alternative structures to rigidize an external working channel.

FIG. 42 illustrates an alternative nested element embodiment.

FIGS. 43-46 illustrate alternative nested element embodiments.

FIGS. 47A-48 illustrate working channel embodiments that utilize electro-active polymers.

FIGS. 49A and 49B illustrate a working channel having a multiplicity of nestable hourglass embodiments.

### DETAILED DESCRIPTION OF THE INVENTION

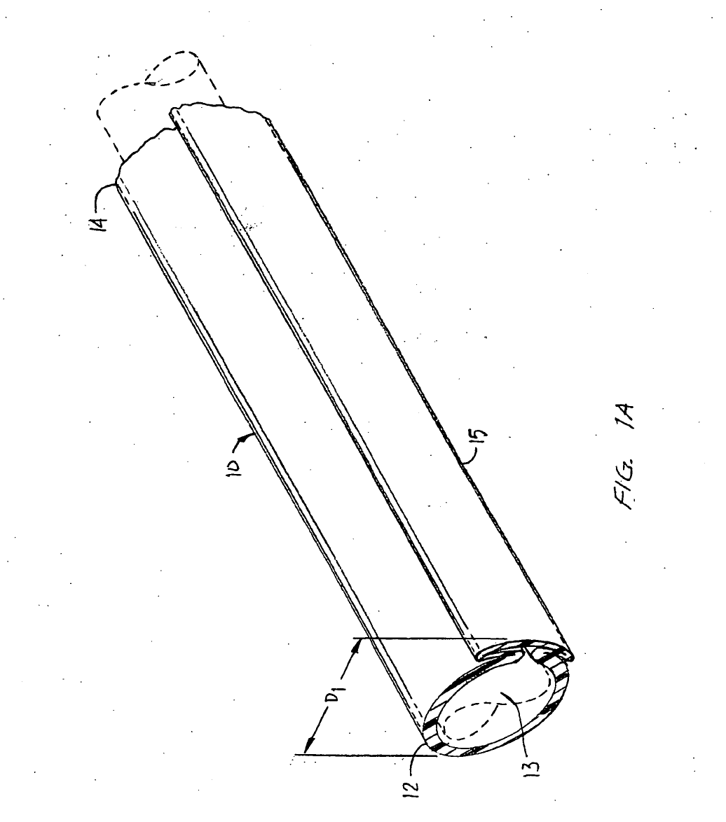

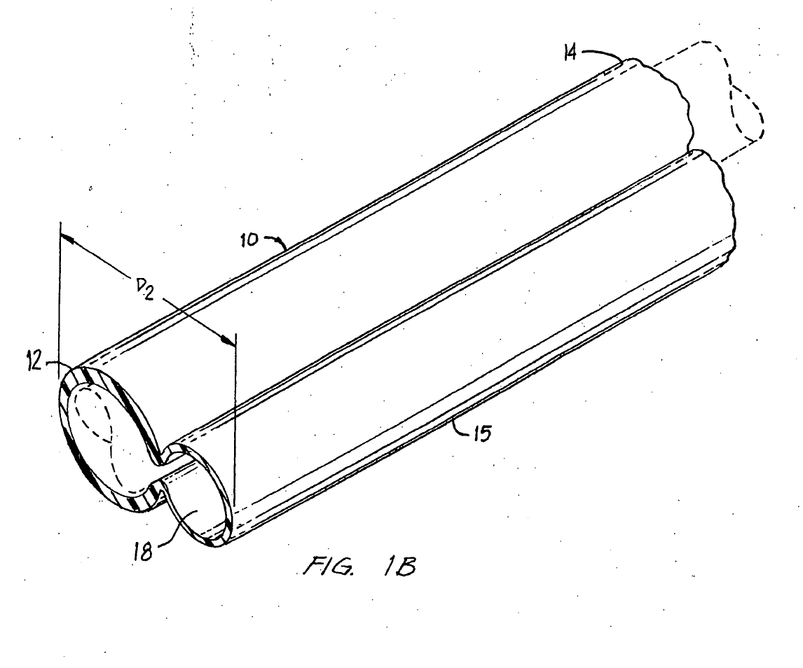

FIG. 1A illustrates an instrument 10 with an expandable channel 15. The instrument is elongate and has a comparatively small effective diameter, and in most embodiments, has a smaller overall cross section area than conventional instruments adapted for the same purpose or task, for reasons set forth below. The instrument 10 may be navigated to a selected site and supports the working channel 15 in both the collapsed (FIG. 1A) and deployed (FIG. 1B) configurations. The instrument typically has a lumen extending therethrough to support fiber optics, bending control components, and other components, depending on such factors as the degree of flexibility required, type of associated channel release mechanism, the constitution material, and the like. The distal tip and shape of the instrument 10 may be tapered and/or straight, curved, round or j-shaped, depending on factors such as physician preference, the anatomy of the tubular organ or region of interest, degree of stiffness required, and the like. Additionally, the tip may also contain a separate device such as a spectroscopic camera, needles, suturing device stapler, and the like. Either or both of the instrument 10 or expandable working channel 15 may include a coil or other suitable element to allow for fluoroscopic or other visualization. The instrument 10 or channel 15 may include one or more radio-opaque markers that indicates the location of the distal section of the delivery guide upon radiographic imaging. Usually, the marker will be detected by fluoroscopy.

In some embodiments, the steerable instrument and/or the expandable working channel may include positioning components to aid in imaging the position and orientation of the endoscope and/or external channel using an external imaging modality. In use, the signal from the positioning element is detected by or used in an external display to provide a real-time—including three-dimensional—view of the position and orientation of the instrument and/or channel within the body. Examples include RFID tags or global positioning system (GPS) elements (e.g., telemeters) adapted for use in the body and with the instrument and/or working channel. In use the location information received from the instrument and/or scope is used in combination with another imaging modality to provide real time integration of the position information to the image. For example, one or more electromagnetic transmission coils or other identifying components may be attached to the instrument and/or channel and used to provide position information. In a specific example, the positioning element is one or more electromagnetic transmission coils provided on the instrument and/or external channel. The signal from the electromagnetic transmission coil positioning element is detected by a low intensity magnetic field to display a real-time three-dimensional view of the position and orientation of the instrument and/or channel. The electromagnetic transmission coils and detection system may be the ScopeGuide 3-D Imager manufactured by Olympus.

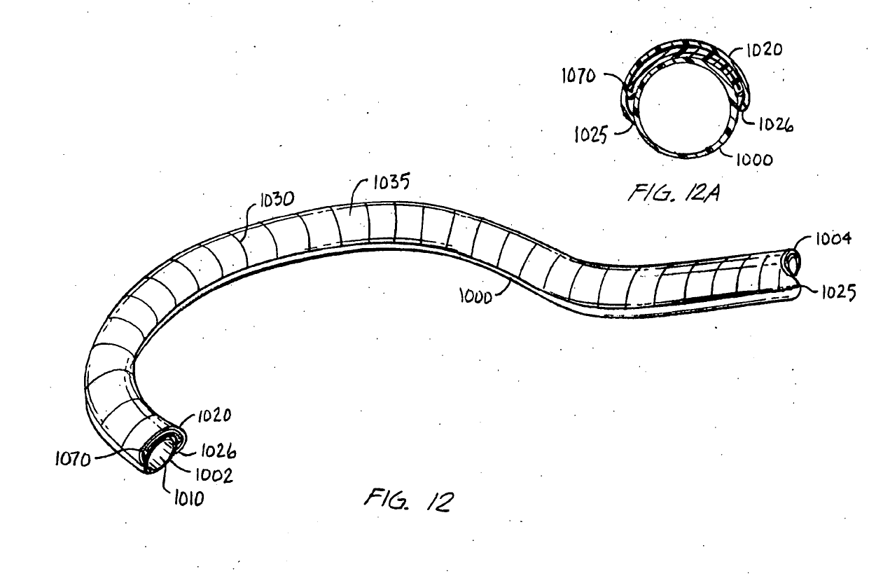

In some embodiments, all or a part of the instrument 10 or working channel 15 may be made from any biocompatible material including, but not limited to, stainless steel and any of its alloys; titanium alloys, e.g., nickel-titanium alloys; other shape memory alloys; tantalum; polymers, e.g., polyethylene and copolymers thereof, polyethylene terephthalate or copolymers thereof, nylon, silicone, polyurethanes, fluoropolymers, poly (vinylchloride), electroactive polymers and combinations thereof. Examples of a combination of materials are the semi-tube embodiments of FIGS. 10-13A.

Physical properties of the instrument and working channel to consider include, but are not limited to: length, diameter of combined instrument and channel when the channel is in a deployed configuration, degree of flexibility, stretchability and lateral stiffness, and the like. These physical properties will be modified to account for such factors as lumen diameter, size of therapy or treatment area, type of luminal structure, or solid organ or tissue involved. It is to be appreciated that the instrument and external working channel concepts described herein are scalable and generally applicable to large hollow body organs such as portions of the colon as well as fine, small diameter vessels in the peripheral vasculature or the brain.

The external working channel 15 is shown in a stowed configuration in FIG. 1A and a deployed configuration in FIG. 1B. The working channel interior volume 18 is visible in FIG. 1B. The working channel is typically formed from polymeric materials. In other embodiments, the external working channel may be formed from a metal. In still other embodiments, the expandable working channel can be made from an inelastic polymer, such as PVC, acrylic, polycarbonate, polyethylene terephthalate or other thermoplastic polyesters. For example, embodiments of the semi-tube working channel described below with regard to FIGS. 10-13A. In other embodiments, the working channel can be made from an elastic, elastomer material.

In additional other embodiments, the external channel is formed from an elastomeric material that is a thermoplastic, elastomeric material, such as polyurethane containing one or more conventional slip agents, such as wax, oil, silicone or silica. Such slip agents are commonly used in the field of elastomeric materials, and an individual having ordinary skill in such an art will understand how to treat the elastomeric material to provide the desired properties for reduced friction both within and about the working channel. The treated elastomeric material allows for small diameter, thin-walled elastic medical components that can be easily, inexpensively, and quickly manufactured. Similar treatments may also be applied for ease of insertion of an instrument. Also, in additional embodiments the elastic material of the channel is made from an elastomeric material treated with slip agents, the channel can be formed such that when in the deployed configuration instruments are more readily inserted into and advanced along the channel lumen.

It is to be appreciated that the slip agents allow instruments to be inserted into the elastic working channel without the instrument distal end binding, catching, or excessively distorting the channel during instrument movement. In still other embodiments, a lubricious coating may be placed on some or all of the surfaces of the instrument or the working channel if desired to facilitate advancement. The lubricious coating typically will include hydrophilic polymers such as polyvinylpyrrolidone-based compositions, fluoropolymers such as tetrafluoroethylene, or silicones. In one variation, the lubricious coating may constitute a hydrophilic gel.

The instrument 10 has a proximate end 12, a distal end 14 and an interior volume or lumen 13 extending along the length of the instrument 10. In this embodiment, the interior volume of channel 15 is in communication with the interior volume 13 of the instrument 10. The instrument 10 and stowed working channel 15 have a diameter of D1. In one embodiment, D1 is less than the diameter of a comparably functional instrument 10 having a fixed size interior working channel. A “comparably functional” instrument is one that is able to perform the same basic functions. It is to be appreciated that the outer surface of the instrument 10 could be attached to the exterior channel 15 or that the external channel 15 could be attached to or integrally formed with a disposable sheath that covers both the instrument 10 and the working channel 15. The endoscope and the deployed working channel have a diameter of D2. D2 is greater than D1.

The instrument 10 could be any medical or industrial instrument. The internal structure and mechanisms within the instrument that provide the instrument's other functions have been omitted for clarity. Exemplary instruments include but are not limited to inspection scopes, endoscopes, colonoscopes, thoracoscopes, neuroscopes, laparoscopes, catheters, guide catheters, trocars, cannulas and the like. The instrument may be similar in functionality to a conventional instrument having an internal working channel. However, the interior volume 13 of instrument 10 is smaller than the internal volume of a comparably functional instrument because the interior volume 13 is reduced by eliminating the working channel from within the interior volume 13. In these embodiments, the conventional fixed size interior working channel is replaced or enhanced by a collapsed, and expandable exterior working channel.

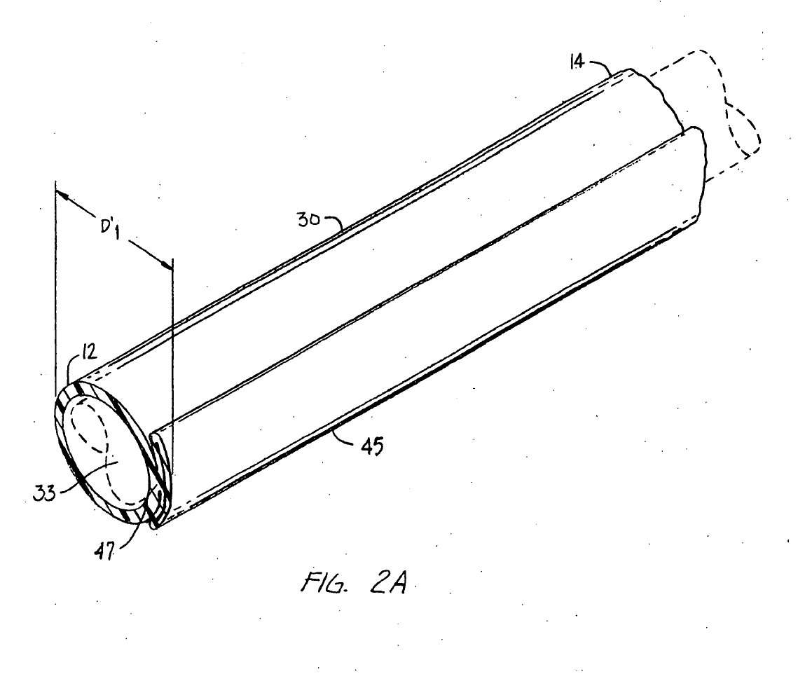

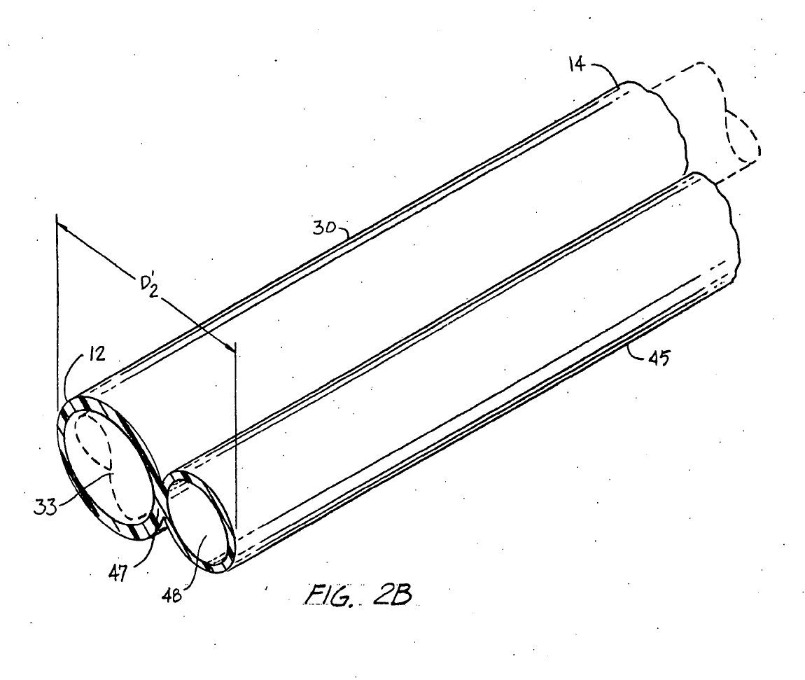

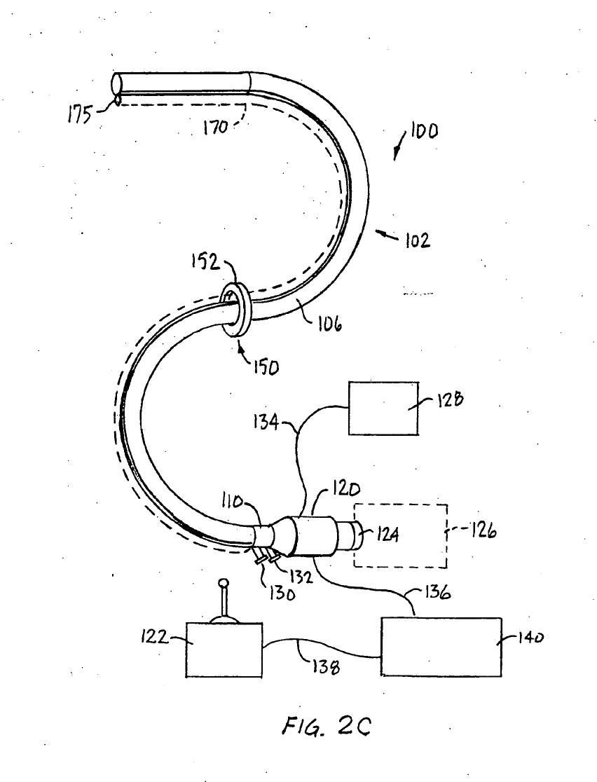

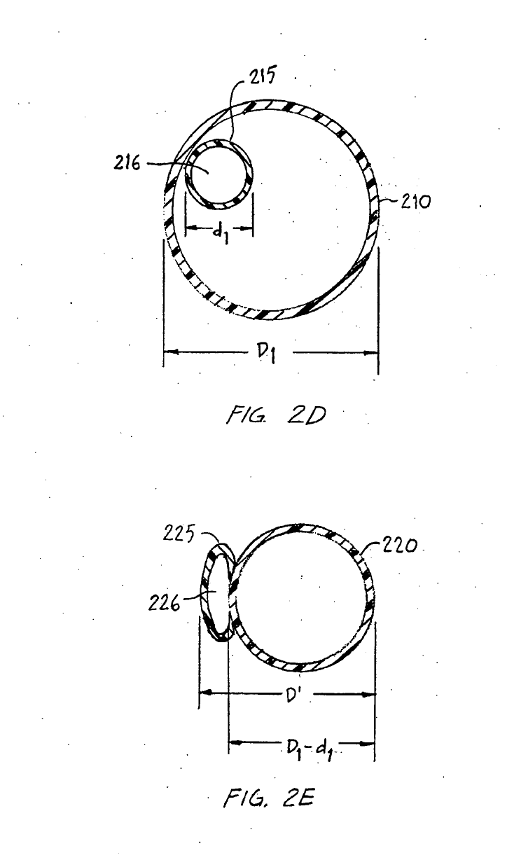

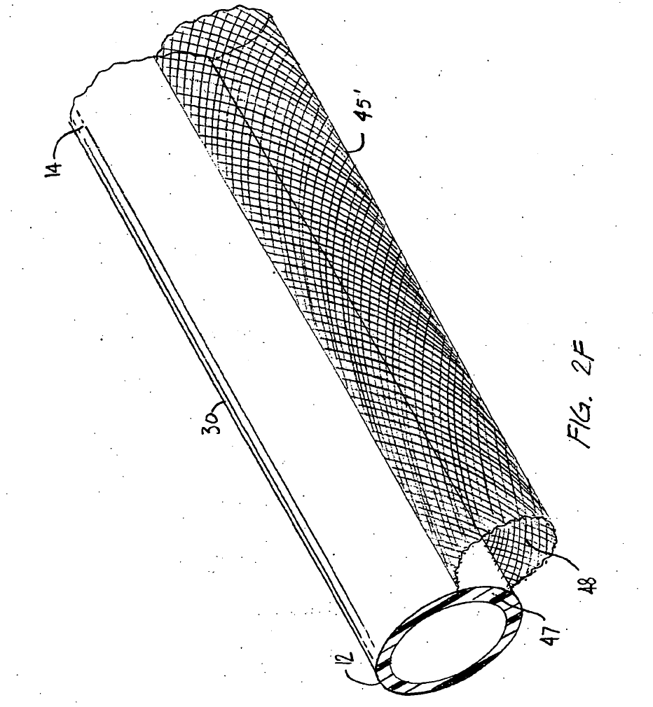

FIG. 2A is an instrument 30 having a proximal end 12, a distal end 14 and a lumen 33 therebetween. The instrument 30 also has an expandable, external, “closed” channel 45. The working channel 45 is illustrated in stowed configuration in FIG. 2A and a deployed configuration in FIG. 2B. The working channel 45 is a “closed” working channel because the channel interior 48 is separated from instrument interior 33 by wall portion 47. The instrument 30 and stowed working channel 45 have a diameter of D′1 (FIG. 2A).

FIG. 2B is the expandable closed channel 45 of FIG. 2A in deployed position. The expandable channel 45 is a closed channel because the interior channel volume 48 is separate from the instrument interior volume 33. The instrument 30 and the deployed working channel 45 have a diameter of D′2. D′2 is greater than D′1. In use, the instruments 10, 30 may navigate, propagate or be advanced while the expanded channel is in a stowed configuration thereby allowing the instrument to navigate in smaller spaces. Once the instrument is positioned, the expandable channel may be positioned into the larger diameter deployed configuration (FIG. 1B, 2B) so that tools, surgical instruments, therapeutic devices, exploratory devices and the like may be advanced along the interior volume 48 of the expandable exterior channel 45.

In some embodiments, the device or devices advance along and exits the distal most portion of the external working channel. In some other embodiments, the external working channel has an opening proximal to the distal opening. This opening allows a device within the external working channel interior volume to pass to a position outside of the external working channel. Additionally, it is to be appreciated that because the working channel volume is not fixed but is instead collapsible and deployable at will and because the working channel is exterior to the rest of the instrument, larger diameter working channels may be provided on instruments having smaller diameters than the size of conventional comparably functional instruments.

Although some embodiments of the working channel of the present invention are illustrated as having solid sidewalls, other sidewall constructions are possible. It is to be appreciated that the construction of the expandable channel may be from virtually any material that meets the operational and functional needs of a particular application. FIG. 2F illustrates one illustrative non-solid sidewall working channel 45′. The working channel 45′ has a mesh sidewall. The mesh sidewall could be formed from metal, plastic, or fabric. Like other working channel embodiments, the non-solid working channel 45′ could also be formed from a material that is treated with a biocompatible coating. Exemplary considerations for additional sidewall materials include size of device or devices to traverse the working channel lumen 48 so that the mesh size of the working channel does not ensnare the device. Another consideration is the ability of the non-solid material to move between stowed and deployed configurations.

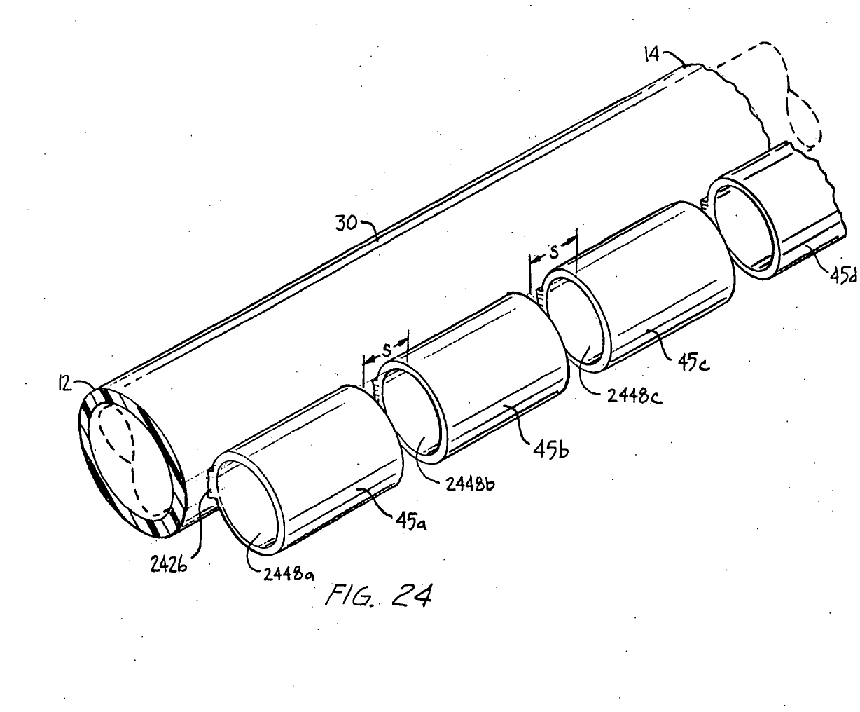

FIGS. 24, 25, and 26 illustrate additional alternative working channel sidewall configurations. In contrast to the continuous sidewall of the working channel 45 in FIG. 2B, the working channel in FIG. 24 comprises a plurality of channel segments 45a, 45b, and 45c and a portion of another segment 45d. Each of the segments are suitably attached to the endoscope 30 at attachment points 2426. Adjacent channel segments are separated by a spacing “S”. The working channel lumen is defined by the lumen of each channel segment, 2448a, 2448b, 2448c, respectively. The spacing “S” is selected so that a tool, instrument or other object exiting the distal end of lumen 2448a will enter the proximal end of the lumen 2448b and so forth. While three complete channel segments are shown, more or fewer segments may be provided. Each of the channel segments is movable between a stowed configuration and a deployed configuration as discussed herein. The segments in FIG. 24 are illustrated in a deployed configuration.

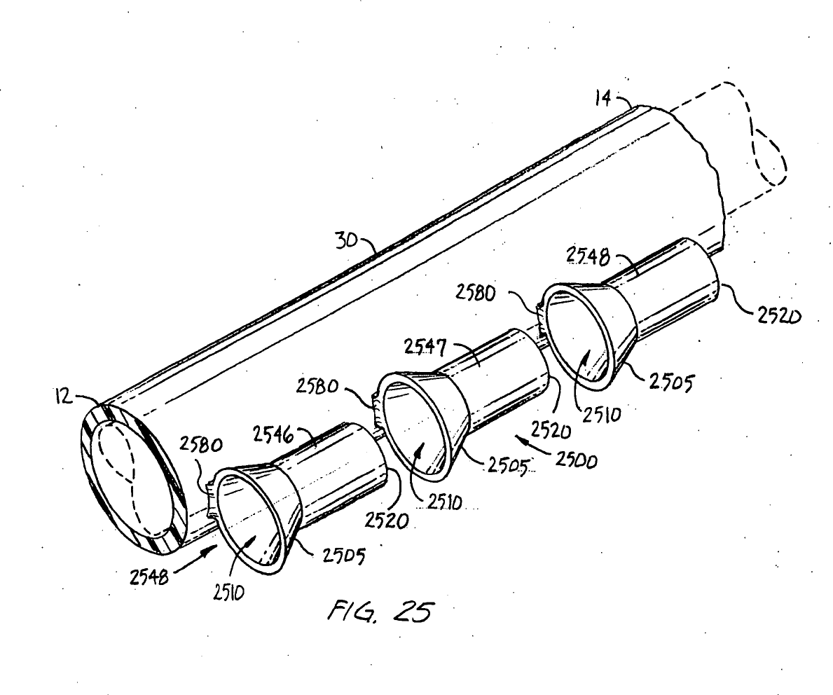

FIG. 25 illustrates an embodiment of a segmented working channel 2500. The segmented working channel 2500, like the segmented embodiment in FIG. 24, comprises a plurality of segments. Each of the channel segments is movable between a stowed configuration and a deployed configuration as discussed herein. In this illustration, there are three segments, namely, 2545, 2546, and 2547, illustrated in a deployed configuration. Each of the segments are suitably attached to the endoscope 30 at attachment points 2580. The specifics of a segmented working channel will be described with reference to segmented working channel 2546. The segmented working channel 2546 has a segment body with a flared proximal end 2505. In the illustrated embodiment, the segmented body is generally cylindrical. A lumen 2548 extends from the proximal opening 2510 to the generally cylindrical distal opening 2520. The opening 2520 may have shapes other than cylindrical and follows the shape of the segment body. The lumen 2548 extends from the distal opening 2520 of one segment to the proximal opening 2510 of the adjacent segment. Moreover, the flared proximal end 2505 has a sloped surface so that an instrument, tool or other device exiting a distal opening 2520 is received and guided towards the cylindrical body interior.

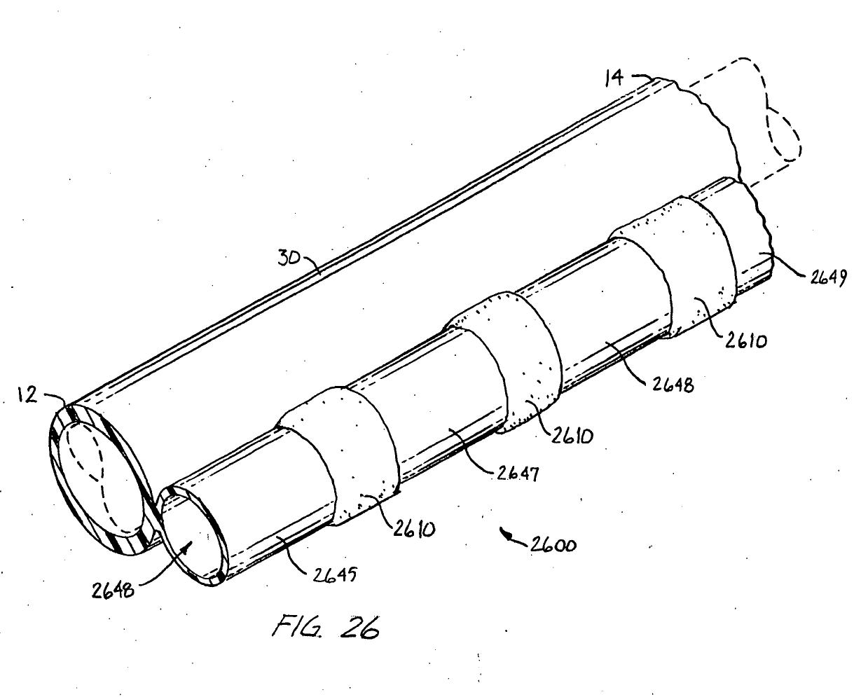

FIG. 26 illustrates another embodiment of a segmented working channel according to another aspect of the invention. The segmented working channel 2600 includes several segments joined by a seal 2610. In this embodiment, segment 2645 is connected to segment 2647 using a seal 2610, segment 2647 is joined to segment 2648 using a seal 2610, and so forth. The seal 2610 is made of a flexible material that provides connectivity between the interiors of each segment. As such, the working channel lumen 2648 includes the interior of each segment and the seal between them. Each of the channel segments and the seal between the segments is movable between a stowed configuration and a deployed configuration as discussed herein. In this illustration, the segments are illustrated in a deployed configuration.

FIG. 2C illustrates an embodiment of an instrument, such as an controllable segmented endoscope as described in U.S. Pat. No. 6,468,203 that has been modified to include an expandable external working channel according to an embodiment of the present invention. U.S. Pat. No. 6,468,203 is incorporated herein by reference in its entirety. FIG. 2C shows a steerable instrument 100 having an external working channel 170 according to one embodiment of the present invention. In this illustrative embodiment, the steerable instrument is a segmented controllable endoscope with a working channel 170 has a lumen 175 that is available when the working channel 170 is in a deployed configuration. The working channel 170 is similar to the external working channel 45 of FIGS. 2A and 2B. The endoscope 100 has an elongate body 102 with a manually or selectively steerable distal portion 104 and an automatically controlled proximal portion 106. The selectively steerable distal portion 104 can be selectively steered or bent up to a full 180 degree bend in any direction. A fiberoptic imaging bundle 112 and one or more illumination fibers 114 extend through the body 102 from the proximal end 110 to the distal end 108. Alternatively, the endoscope 100 can be configured as a video endoscope with a miniaturized video camera, such as a CCD camera, positioned at the distal end 108 of the endoscope body 102. The images from the video camera can be transmitted to a video monitor by a transmission cable or by wireless transmission. Optionally, the body 102 of the endoscope 100 may include one or two instrument channels 116, 118 that may also be used for insufflation or irrigation. The body 102 of the endoscope 100 is highly flexible so that it is able to bend around small diameter curves without buckling or kinking. When configured for use as a colonoscope, the body 102 of the endoscope 100 is typically from 135 to 185 cm in length and less than about 15 mm in diameter in one embodiment, between 10 to 15 mm in diameter in another embodiment and less than 10 mm in diameter in yet another embodiment. The endoscope 100 can be made in a variety of other sizes and configurations for other medical and industrial applications.

A proximal handle 120 is attached to the proximal end 110 of the elongate body 102. The handle 120 includes an ocular 124 connected to the fiberoptic imaging bundle 112 for direct viewing and/or for connection to a video camera 126. The handle 120 is connected to an illumination source 128 by an illumination cable 134 that is connected to or continuous with the illumination fibers 114. A first luer lock fitting 130 and a second luer lock fitting 132 on the handle 120 are connected to the instrument channels 116, 118.

The handle 120 is connected to an electronic motion controller 140 by way of a controller cable 136. A steering control 122 is connected to the electronic motion controller 140 by way of a second cable 138. The steering control 122 allows the user to selectively steer or bend the selectively steerable distal portion 104 of the body 102 in the desired direction. The steering control 122 may be a joystick controller as shown, or other known steering control mechanism. The electronic motion controller 140 controls the motion of the automatically controlled proximal portion 106 of the body 102. The electronic motion controller 140 may be implemented using a motion control program running on a microcomputer or using an application-specific motion controller. Alternatively, the electronic motion controller 140 may be implemented using a neural network controller.

There is also provided a working channel controller 128 connected to the handle 120 and working channel 170 via connector 134. The working channel controller 128 allows the user to, for example, release a stowed channel into an expanded position, selectively release portions of a multi-channel embodiment, and return a deployed channel to a stowed condition. The working channel controller 128 and connector 134 are modified as needed to control the type of channel used as well as the type of release or deployment methodology. For example, if the expandable channel was deployed by inflating the channel or a hollow channel sidewall, then the working channel controller would include suitable controls for the controlled introduction of fluid or air into the hollow channel via a suitably modified connector 134. Alternatively, if the working channel relied on a mechanical release to transition from a stowed to a deployed condition then the controller 128 and connector 134 would be modified to a mechanical control and connector as would be conventionally used. It is to be appreciated that a wide array of working channel release techniques and mechanisms may be used, including but not limited to: magnetic, electric, electronic, electromagnetic, electrolytic, hydraulic, pressure based (i.e., pressure increase to deploy, pressure decrease to stow), shape memory alloys, electroactive polymers, springs, latches, cable pulls, and the like.

An axial motion transducer 150 is provided to measure the axial motion of the endoscope body 102 as it is advanced and withdrawn. The axial motion transducer 150 can be made in many possible configurations. By way of example, the axial motion transducer 150 in FIG. 2 is configured as a ring 152 that surrounds the body 102 of the endoscope 100. The axial motion transducer 150 is attached to a fixed point of reference, such as the surgical table or the insertion point for the endoscope 100 on the patient's body. As the body 102 of the endoscope 100 slides through the axial motion transducer 150, it produces a signal indicative of the axial position of the endoscope body 102 with respect to the fixed point of reference and sends a signal to the electronic motion controller 140 by telemetry or by a cable (not shown). The axial motion transducer 150 may use optical, electronic, magnetic, or mechanical means to measure the axial position of the endoscope body 102.

FIGS. 2D and 2E illustrate how the diameter of an instrument may be reduced by using an external channel according to an embodiment of the present invention. FIG. 2D illustrates a conventional instrument 210 having a conventional fixed diameter internal working channel 215. The remaining interior portion of instrument 210 is devoted to other functional elements (not shown). The conventional fixed diameter internal working channel 215 has a constant internal area 216 and diameter d1. The instrument 210 has a diameter D1. FIG. 2E illustrates a modified instrument 220 having comparable functionality to instrument 210 but having a diameter that is smaller than the diameter of the conventional instrument 210. The diameter of the modified working instrument 220 is less than the conventional instrument 210 because the modified instrument 220 has no fixed size internal working channel. Instead, the fixed size working channel has been removed from the interior of the instrument 220 (leaving the other interior functional elements (not shown)) and the diameter of the instrument 220 reduced accordingly. The instrument 220 utilizes an embodiment of the external working channel 225 having a stowed or compressed area 226 that may be smaller than illustrated. As described elsewhere, the external channel lays flat against the exterior wall of the instrument. The diameter D′ will be only slightly greater than the diameter d2 of the main portion of the instrument.

One advantage of embodiments of the present invention is that the instrument size may be decreased by removing the interior fixed volume working channel and replacing the working channel functionality with a collapsed but expandable exterior working channel. Instruments without a fixed size interior working channel may have smaller overall diameters while navigating along a pathway to reach an objective compared to conventional instruments of comparable functionality.

After completing the navigation to an objective, the expandable working channel can released from the stowed position into a deployed position thereby making the working channel available for use. Thereafter, the instrument may continue navigation with the working channel deployed or the working channel may be returned to the stowed condition prior to resuming navigation.

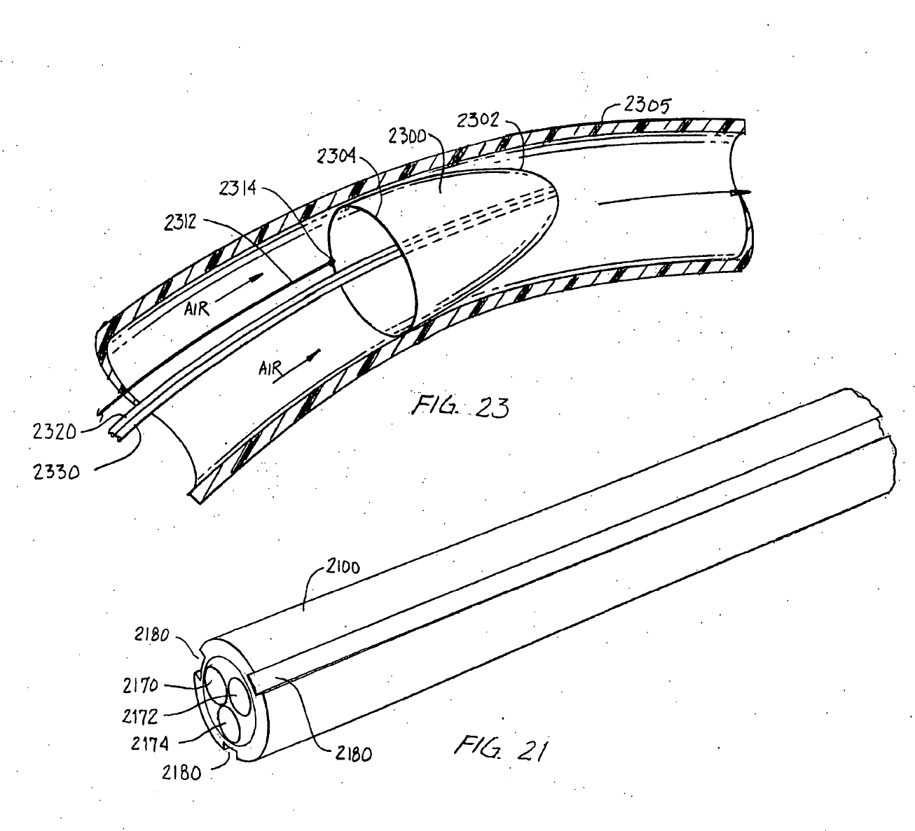

Alternatively, rather than returning a deployed working channel to a stowed configuration for removal, a deployed external working channel may be detached from the steerable instrument and removed separately. The external working channel may be releaseably attached to the steerable instrument using any of a wide variety of conventional attachment methods. Consider the exemplary removable working channel 43 in FIGS. 2G, 2H and 21. The removable working channel is illustrated in a deployed position in FIG. 2G much like working channel 45 in FIG. 2B. In contrast to working channel 45 that is attached to the steerable instrument 30 using a solid connector 47, the removable working channel 43 is attached using a pull cord 62. The pull cord 62 extends along the length of the channel 43 with features 64 that match apertures 61 forming an attached connection 66 (FIGS. 2G and 2I). To detach the channel 43 from the instrument 14, the cord 62 is pulled in a proximal direction. As the cord moves proximally, the features 64 separate from the apertures 61 and release the channel 43 (FIG. 2H). In one embodiment, the expandable channel 43 is configured to evert as it is separated from the instrument 30 and removed.

While FIG. 2G illustrates a single detachable external working channel, a steerable instrument may have more than one detachable working channel. In one alternative embodiment, a plurality of releasable channels may be arranged about a steerable instrument and then used as needed during an examination performed with the steerable instrument. For example, an exemplary steerable instrument has 4 stowed releasable working channels 43. With all four channels 43 in a stowed configuration the instrument is advanced to the first therapeutic site where a procedure is performed using a first working channel 43. At the conclusion of the first procedure, the deployed releasable channel 43 is removed using releasing means suited to the channel 43. For example a pull cord as illustrated in FIG. 2H. Thereafter, the steerable instrument advances to the site of the next procedure. A second channel 43 is deployed providing a working channel for the next procedure. Once the next procedure is completed the second channel 43 is detached from the controllable instrument and removed. The process of deploying, using, detaching and removing a releasable channel 43 repeats until the procedures are completed or the supply of releasable channels 43 is exhausted.

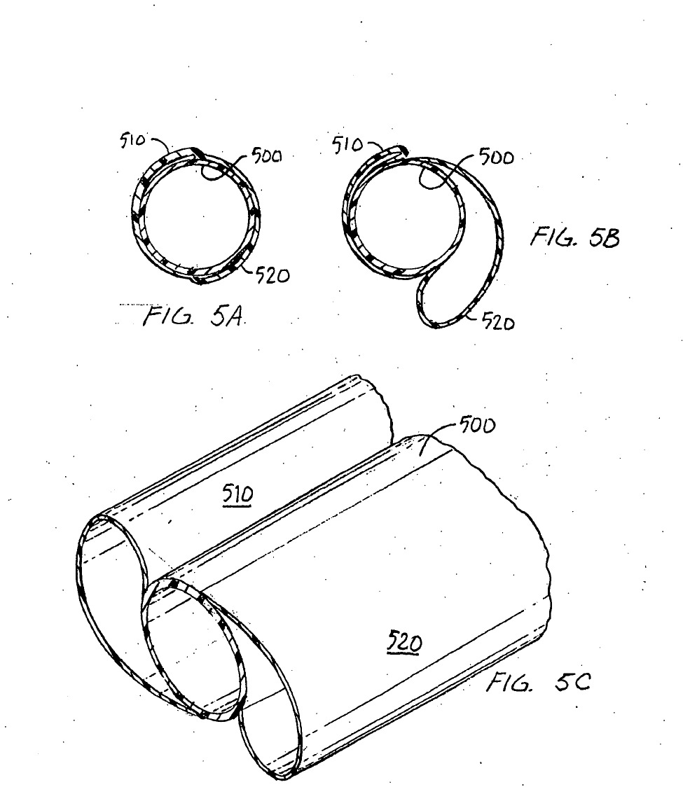

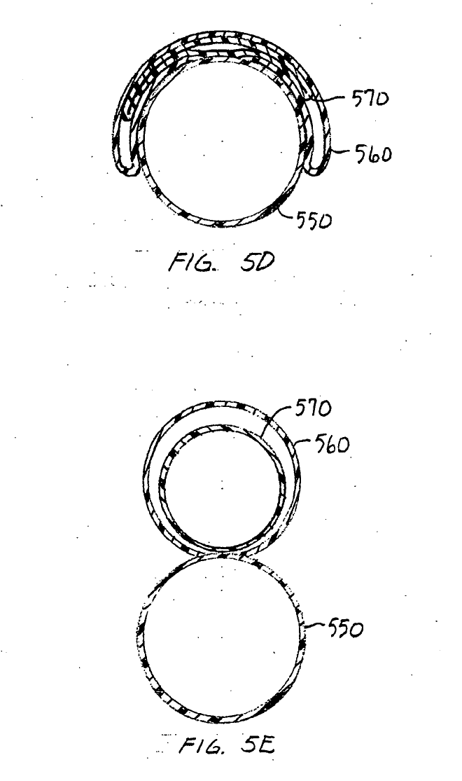

It is to be appreciated that embodiments of the invention may also be used in combination with conventional instruments such as instrument 210 in FIG. 2D. A conventional instrument 210 need not be altered to remove its internal working channel 215 to realize the benefits of the invention. Consider the example where an instrument 210 is to include a second working channel of the same size as channel 215. If added conventionally, then the additional channel would be added within the interior of instrument 210 and likely require that the instrument diameter D1 be enlarged to accommodate the additional fixed diameter channel. In contrast, consider an embodiment where the instrument 210 is modified to include the desired additional channel external to the instrument. There would be only a slight increase in overall diameter to provide for the stowed external working channel. Alternatively, the conventional instrument 210 desiring an additional working channel could be modified according to some of the multi-channel embodiments described herein (e.g., the embodiments of FIG. 5, 6, or 7).

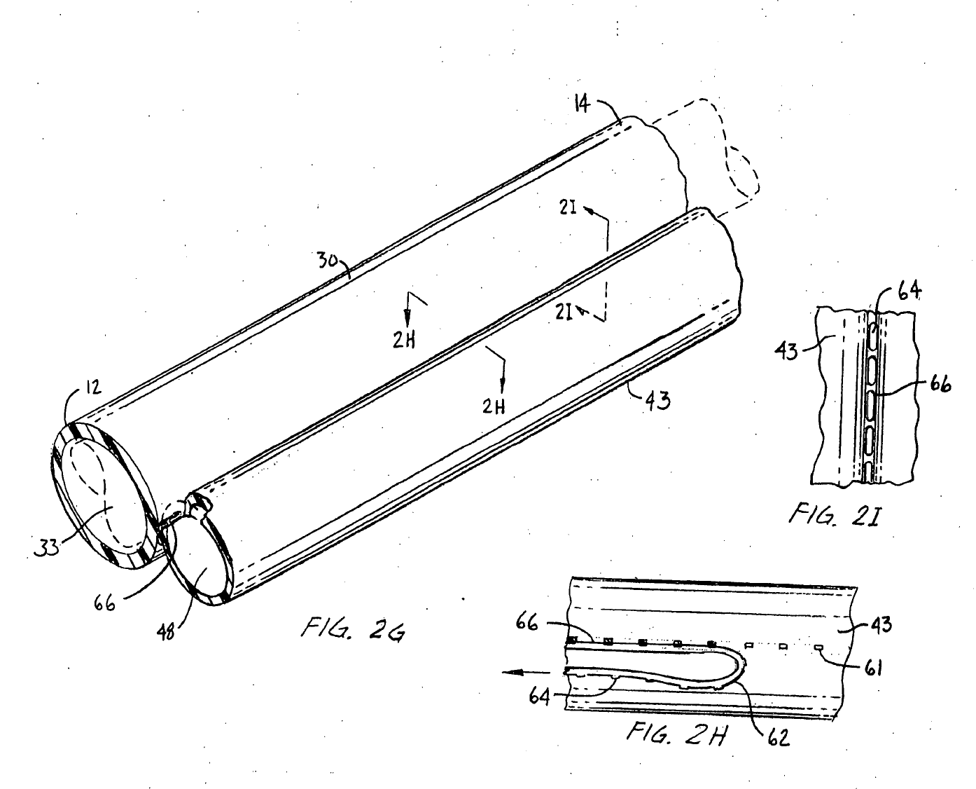

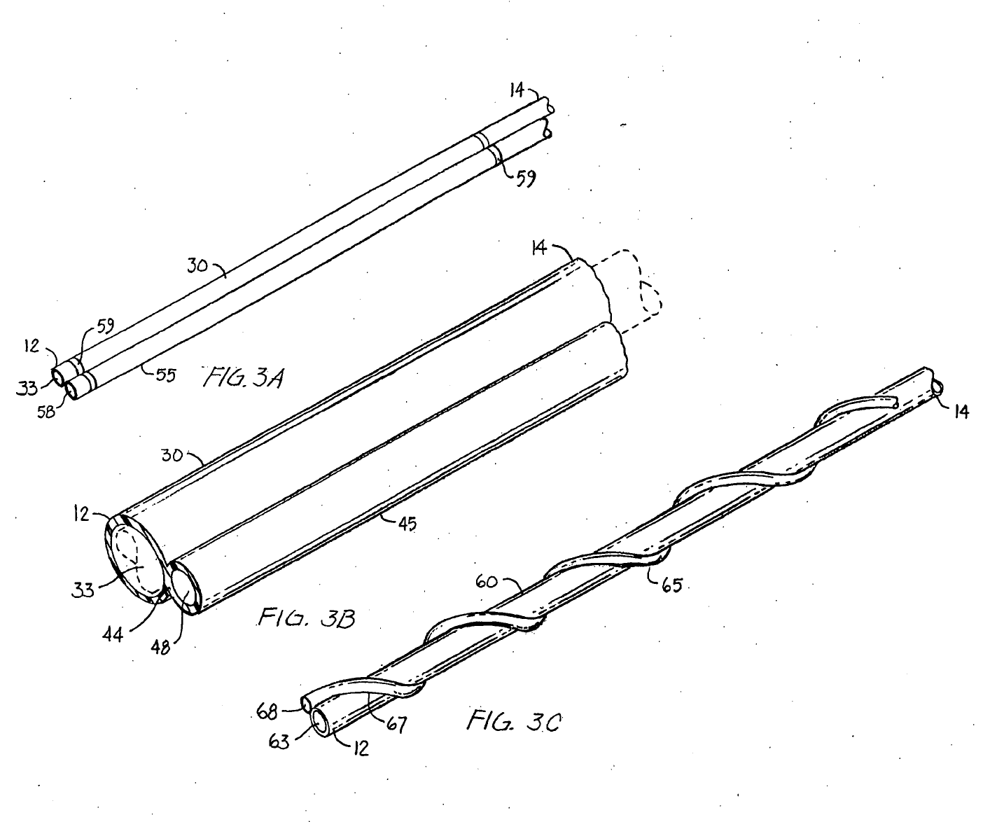

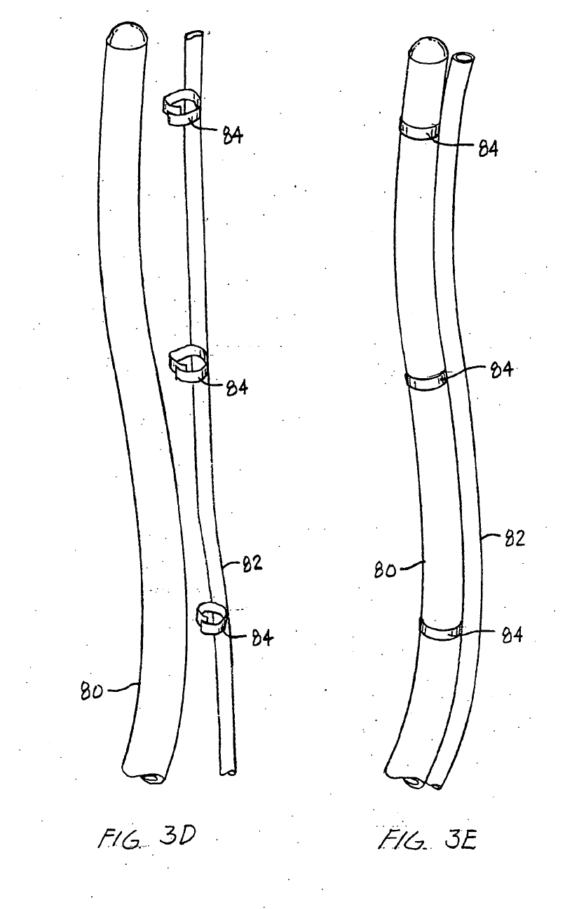

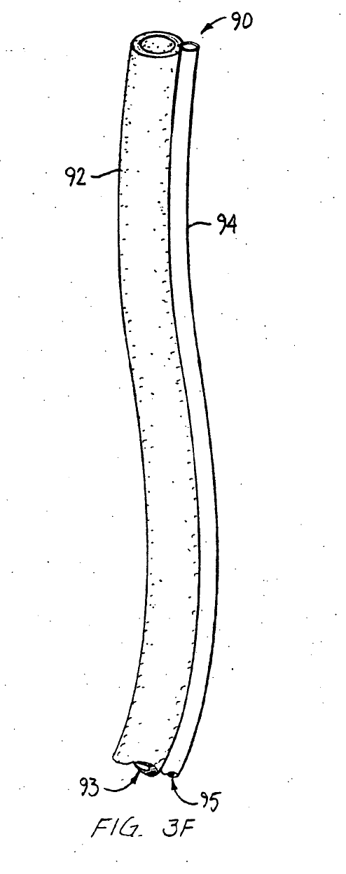

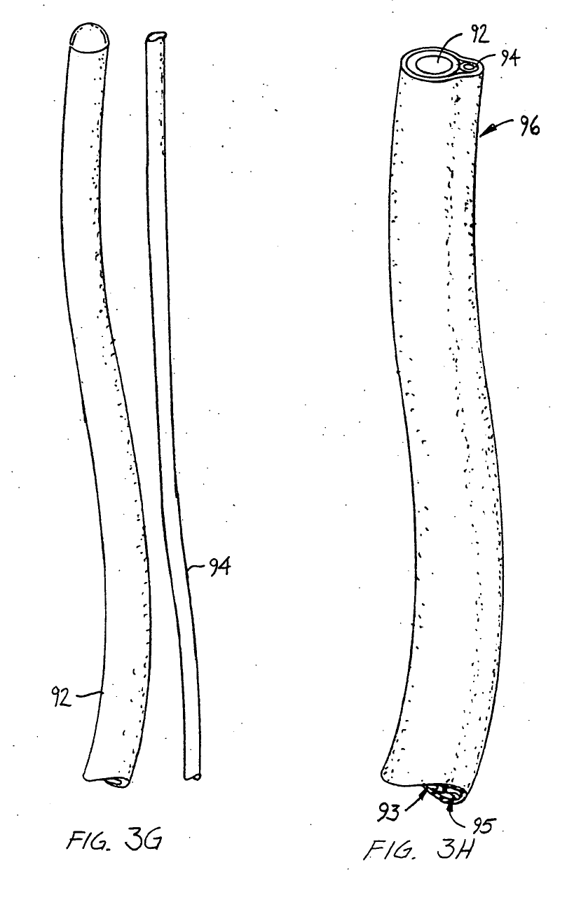

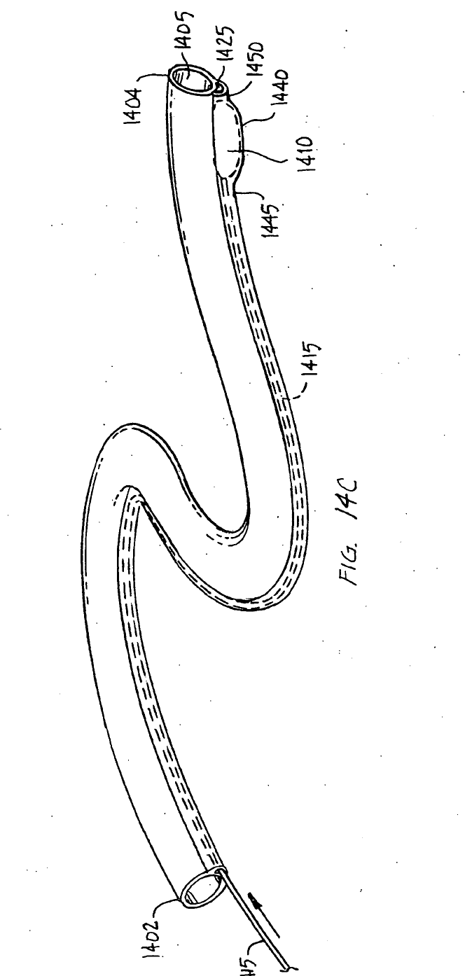

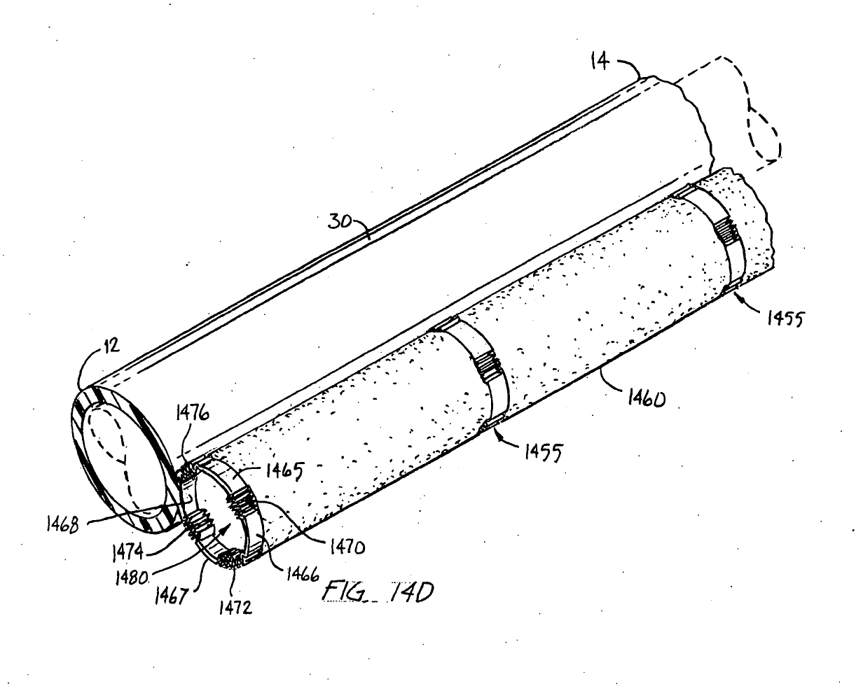

FIGS. 3A, B and C show some alternative relationships between an endoscope and a deployable working channel. FIG. 3A shows discrete attachments 59 along the length of the instrument. In contrast, 3B illustrates connection 44 along the length of the instrument at a constant radial position, here along the side at the mid-radial or 3 o'clock position. In contrast, FIG. 3C illustrates a steerable instrument 60 having a proximal end 12, a distal end 14 and a lumen 63 therethrough. An expandable working channel 65 with a lumen 68 is attached to the instrument 60 at various radial connections 67. In the illustrative embodiment of FIG. 3C the expandable working channel is illustrated in a deployed configuration and in a helical pattern about the instrument 60. Others configurations are possible. For example, the channel may form a sinusoidal shape along one side of the endoscope, remaining between the 12 o'clock and 6 o'clock positions. In another alternative embodiment, the external working channel 65 is a deformable channel such as those described below with reference to FIGS. 14A-C.

FIGS. 3D, 3E and 3F provide two alternative illustrative embodiments of the advantageous use of an external working channel of the present invention with an endoscope. FIG. 3D illustrates an endoscope 80 and a detached working channel 82. The detached working channel 82 includes a plurality of fasteners 84 that are used to attach the working channel 82 to the endoscope 80. Three fasteners 84 are illustrated, and more or fewer may also be used. The fasteners 84 may use any known attachment method to secure the working channel 82 to the endoscope 80. In still another alternative embodiment, the external working channel may be formed as part of a sheath adapted to fit on an endoscope.

FIG. 3F illustrates a sheath 90 having an endoscope covering portion 92 and a working channel portion 95. The endoscope covering portion has a lumen 93 sized and adapted to receive an endoscope. The working channel portion 94 has a lumen 95 and is illustrated in a deployed configuration. The working channel portion 94 also has a stowed configuration (not shown). It is to be appreciated that embodiments of the working channel portion 94 may be configured as described in other working channel embodiments. For example, the working channel 94 may be compact but stretchable working channel as described below with reference to FIGS. 14A, 14B and 14C.

In still another alternative embodiment, the external working channel 94 and endoscope 92 may be separate components held together by an external sheath 96. The working channel 94 is positioned against endoscope 92 (FIG. 3G). The external channel 94 is held in place using a sheath 96 that wraps around both the endoscope 92 and the working channel 94. The sheath 96 is formed from a suitable bio-compatible material that is sized to slide over, fit, shrink fit, elastically fit, wrap or otherwise be adapted to hold the working channel 94 along side the endoscope 92 (FIG. 3H). The sheath 96 provides an smooth, slideable, external surface for navigation and movement within the body, as described herein or known to those of ordinary skill in the medical arts.

Advantageously, embodiments of the working channel of the present invention enable a new series of procedures where the screening/diagnosis function is separated from the therapeutic function. Consider an example of a screening instrument. A screening instrument is a steerable or otherwise controllable instrument of reduced size adapted to perform screening and/or diagnostic procedures. The screening instrument may have visualization capabilities, lighting capabilities and/or sensors or devices used to evaluate, measure, image or otherwise obtain information regarding adjacent body portions or surroundings. Because the present invention provides working channel functionality as needed using the techniques described herein, the screening instrument may have no working channel or, alternatively, have only a size restricted working channel.

In use, the screening instrument is used to visualize, evaluate measure, image or otherwise obtain information regarding a body portion or surroundings. Next, if needed, an embodiment of the working channel of the present invention is provided where desired to perform a surgical, diagnostic or therapeutic procedure using the screening instrument as the delivery and/or control and positioning platform for one or more working channel embodiments. As is made apparent in the discussion herein, the screening instrument may be adapted in any number of ways described herein for providing one or more embodiments of the working channel of the present invention.

The following specific examples further illustrate the concept of separating the screening/evaluation function from therapeutic/surgical treatment functions and the use of a screening instrument adapted for providing working channels as and when needed. Consider examination/screening and related therapies for the colon. In this example, a pediatric colonoscope is used as a screening colonoscope for evaluating an adult colon. This screening colonoscope is adapted to deliver an external working channel of the invention as discussed herein but may have a working channel included within its primary lumen. The pediatric-size screening colonoscope is used as an adult exploratory instrument and delivery mechanism for an external working channel.

A pediatric colonoscope or an upper endoscope is a fraction of the size (i.e., about half the diameter) of an adult colonoscope. When an external working channel is attached in a stowed configuration to the outer wall of a pediatric-size screening colonoscope, the small diameter of the screening colonoscope is increased only slightly by the thickness of the stowed working channel. Moreover, the screening instrument with stowed working channel has a smaller diameter than a conventional adult colonoscope without sacrificing any of the screening functionality of an adult colonoscope. The visualization system and support systems (irrigation, insufflation etc.) of the screening colonoscope act as an exploratory instrument in the adult colon. If during or after examination, a surgical, therapeutic or diagnostic procedure to be performed requires a working channel, then a working channel of the present invention is provided, deployed and utilized as needed. If, however, no working channel is needed then the adult patient will have had a colon screening performed using a pediatric colonoscope, likely with much greater comfort but with no loss in efficacy. The same would be true for screening of other portions of the gastrointestinal tract or other parts of the body.

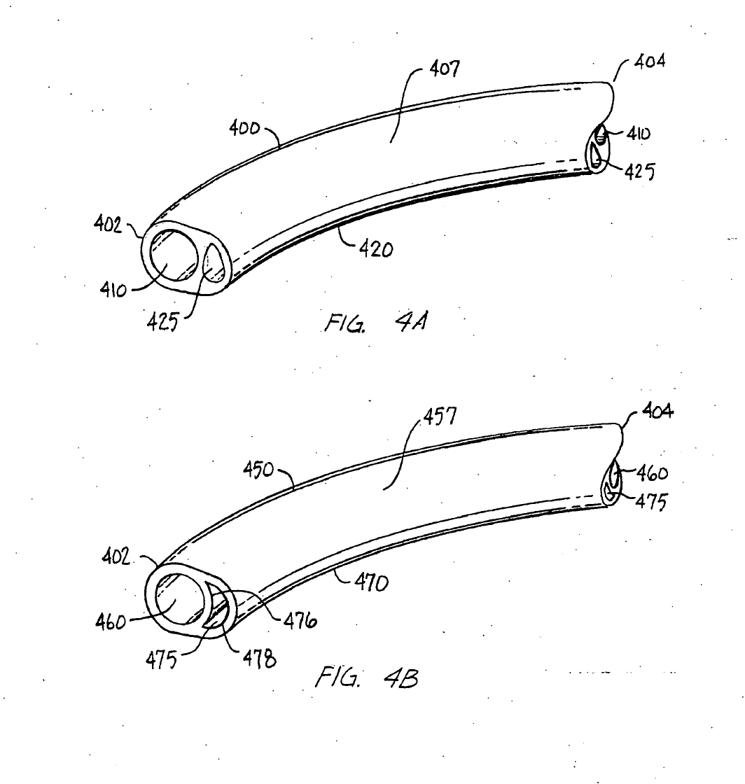

It is also to be appreciated that the cross section area of a working channel of the present invention need not have the same cross section area of the endoscope or instrument used to deliver the working channel. Depending upon channel deployment and delivery techniques (i.e., inflation, release, controlled release, external sidewall, external rail, cable pull, etc.) the shape and dimensions of the working channel may be advantageously altered and reconfigured. FIGS. 4A and 4B illustrate two alternative embodiments having different shaped working channel lumens. The instrument 400 in FIG. 4A has a proximate end 402, a distal end 404 and a lumen 410 extending there between. An external working channel 420 is shown in a deployed configuration so that the working channel lumen 425 extends along the length of the instrument 400. The working channel 420 has a lumen 425 that is semi-elliptical or teardrop in shape. As such, the working channel lumen 425 illustrates that the shape of the working channel lumen need not conform to either the external shape of the instrument or the external shape of the working channel. Instead of conforming to surrounding geometry, the shape of the lumen 425 is advantageously selected to support the procedures performed using or the shape/size of instruments passing along the lumen 425. In other embodiments, the shape of one or both or a portion of the sides of the working channel lumen may conform, for example, to the shape of a portion of the instrument outer surface or the outer shape of the working channel. The instrument 450 has a working channel 470 with such a lumen (FIG. 4B). The working channel lumen 475 has a part of the lumen 476 that conforms to the shape of the instrument lumen 460 while another lumen portion 487 conforms to the shape of the working channel 470. The instruments 400, 450 also illustrate how the expandable, external working channel may be integrally formed from a single cover or sheath that covers both the instrument and the expandable working channel. When contrasted with the exterior appearance of instrument 30 in FIG. 3B, the continuous shape of the external surfaces 407, 457 is made clear.

As indicated above, embodiments of the present invention are not limited to a single expandable working channel. Depending upon application and use, there may be provided multiple expandable, exterior working channels. FIG. 5A-5E illustrate two alternative multi-channel embodiments. FIGS. 5A-5C illustrate an instrument 500 having two separately releasable working channels, 510, 520 that may be deployed individually and independently. FIG. 5A illustrates the channels 510, 520 in stowed position against the exterior walls of the instrument 500. FIG. 5B illustrates a state where the channel 520 is deployed and the channel 510 is stowed. FIG. 5C illustrates both channels 510, 520 in deployed position.

In contrast to FIG. 5A-C where the additional channels are radially separated about the instrument, the instrument 550 has multiple working channels 560,570 in a single radial position (FIG. 5E), an interior working channel 570 and an exterior working channel 560. The channels 560, 570 are illustrated in a stowed condition in FIG. 5D and a deployed condition in FIG. 5E. While illustrated with an interior channel 570 having a diameter almost as large as the exterior channel 560, that need not be the case. The relative size of the internal channel 570 with respect to the external working channel may be varied. In some embodiments, the internal channel is more than half the diameter of the external channel. In another embodiment, the internal channel diameter is about half the size of the external channel. In another embodiment, the internal channel diameter is less than half the diameter of the external channel. In alternative embodiments, more than one pair of concentric expandable channels is arrayed about the instrument 550.

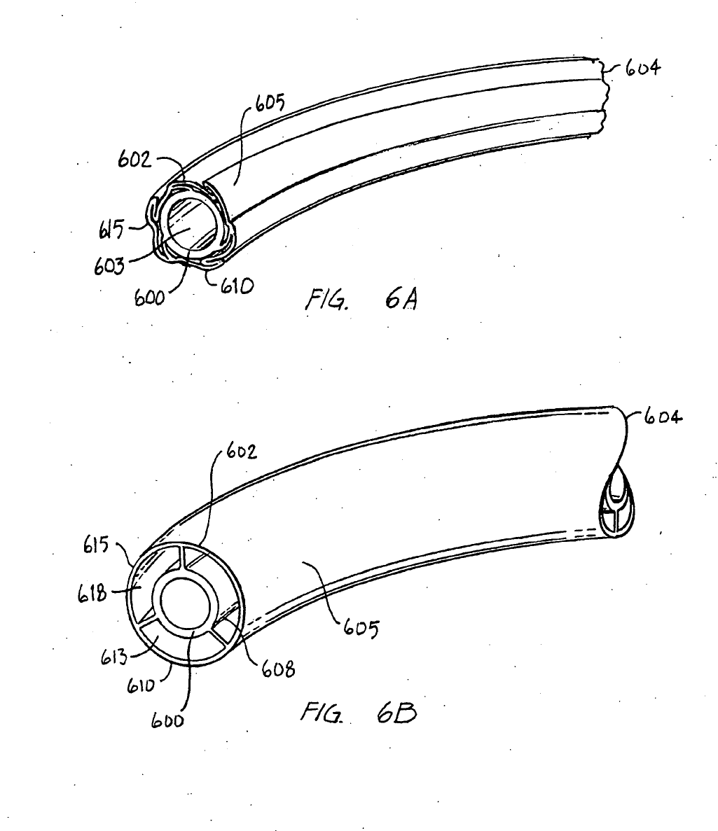

Another multiple working channel embodiment is illustrated in FIGS. 6A, 6B. The instrument 600 has an elongate body with a proximal end 602, a distal end 604 and an internal lumen 603 therebetween. The instrument 600 includes three working channels 605, 610, 615 that together encircle the instrument 600. The channels 605, 610, and 615 are showed in a stowed configuration in FIG. 6A. The channels 605, 610 and 615 are shown in a deployed configuration in FIG. 6B. One advantage of this embodiment is that all three channels are deployed simultaneously to provide working channel lumens 608, 613, 618 that extend from the distal end 604 to proximal end 602 of the instrument 600. It is to be appreciated that the instrument may translate to a site of interest or navigate along a pathway with the channels in a stowed configuration (FIG. 6A). In this configuration the instrument 600 has a smaller diameter and will be easier to navigate into smaller spaces than in the deployed configuration. Once the instrument is positioned in a desired location or if one or more of the working channels are needed, then the instrument 600 is reconfigured into an instrument having one or more working channels (FIG. 6B). In an alternative embodiment, the channels may be configured to be separately deployed rather than having all the working channels formed in a single motion as in the embodiment of FIG. 6A/6B. Three working channels are shown for purposes of illustration only, more or fewer channels may also be used.

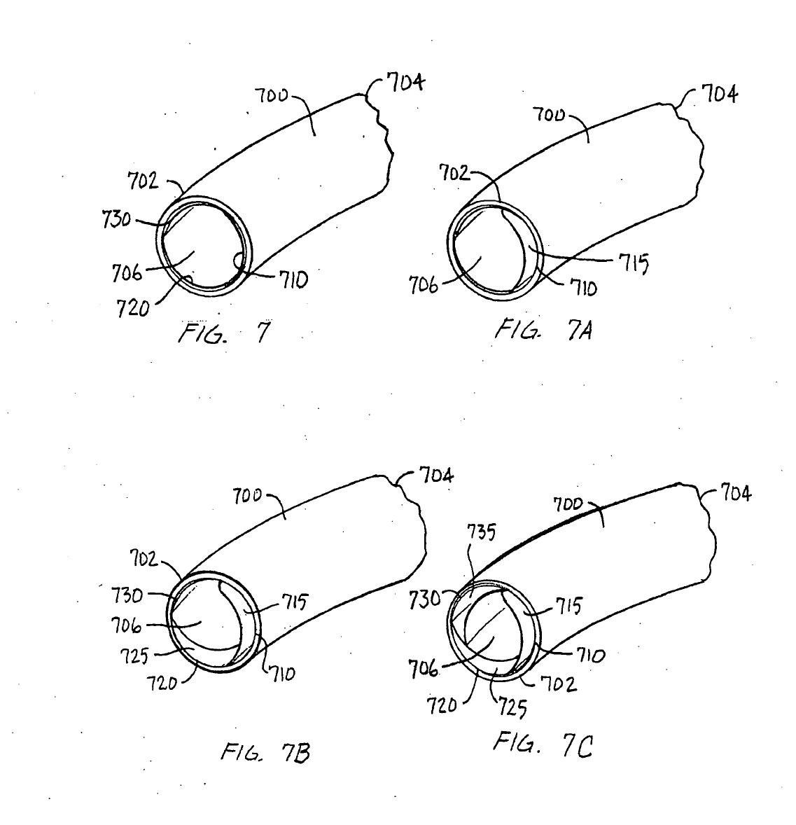

In contrast to an embodiment where all of the external working channels in a multi-channel embodiment are formed simultaneously, there are other multi-channel embodiments where each of the channels may be formed independently or one at a time using controlled release. Instrument 700 includes an elongate body with a proximal end 702, a distal end 704 and a lumen 706 extending there between. Three independently deployable or controlled release working channels 710, 720 and 730 are provided about the instrument 700 exterior. The external working channels are illustrated in a stowed configuration in FIG. 7. The working channel 710 is illustrated in a released or deployed configuration in FIG. 7A. When channel 710 is in a deployed configuration, a working channel or lumen 715 is formed from the proximate end 702 to the distal end 704. The working channel 720 is illustrated in a released or deployed configuration in FIG. 7B. When channel 720 is in a deployed configuration, a working channel or lumen 720 is formed from the proximal end 702 to the distal end 704 in addition to working channel 715. The working channel 730 is illustrated in a released or deployed configuration in FIG. 7C. When channel 730 is in a deployed configuration, a working channel or lumen 735 is formed from the proximal end 702 to the distal end 704, in addition to the channels 715, 725. While FIG. 7C illustrates an embodiment where all three channels are released, that need not be the case. Moreover, the channels may be released in any order and with one or more remaining in a stowed configuration. It is to be appreciated that embodiments of the present invention are moveable between stowed and deployed configurations repeatedly if needed. As such, in a single procedure, an instrument may have numerous configurations or switch between configurations numerous times such as a configuration with no channels deployed, only one channel deployed, only one channel stowed or no channels stowed among others.

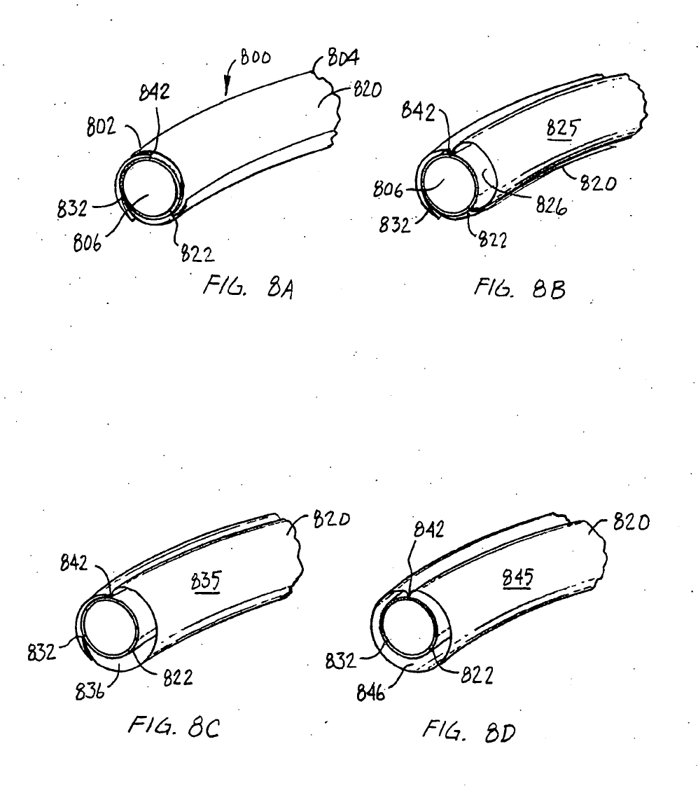

In contrast to embodiments where a working channel release or deploy operation provides additional individual working channels, there are embodiments of the present invention where a working channel release or deploy operation increases the size of a working channel. As such, instead of a controlled release providing separate working channels, a controlled release may be used to create a single working channel having different sizes. This concept is illustrated by instrument 800 in FIGS. 8A-8D.

FIG. 8A illustrates an instrument 800 having an elongate body with a proximal end 802, a distal end 804 and a lumen 806 therebetween. A variable size, controlled release external working channel 820 surrounds the instrument 800. The variable size controllable release working channel 820 is attached to the instrument at attachment points 822, 832 and 842. The working channel 820 is illustrated in a stowed configuration in FIG. 8A. A working channel 825 with a lumen 826 is formed when the working channel 820 is deployed between the attachment points 842 and 822 (FIG. 8B). The variable size working channel 820 remains in a stowed configuration between attachment points 822 and 832. A working channel 835 with a lumen 836 is formed when the variable size working channel 820 is deployed between the attachment points 842 and 832 (FIG. 8C). In this embodiment, the working channel 835 is formed by releasing the attachment point 822. The variable size working channel 820 remains in a stowed configuration between the attachment points 832 and 842. The working channel lumen 836 is larger than the working channel lumen 826. A working channel 845 with a lumen 846 is formed when the variable size working channel 820 is fully deployed and attached only at attachment point 842 (FIG. 8D). In this embodiment, the working channel 845 is formed by releasing the attachment point 832. The working channel lumen 846 is larger than the working channel lumens 826 and 836. In another alternative release procedure, two working channels may be formed by deploying channel 825 and another channel provided between attachment points 842 and 832. Other release procedures are possible.

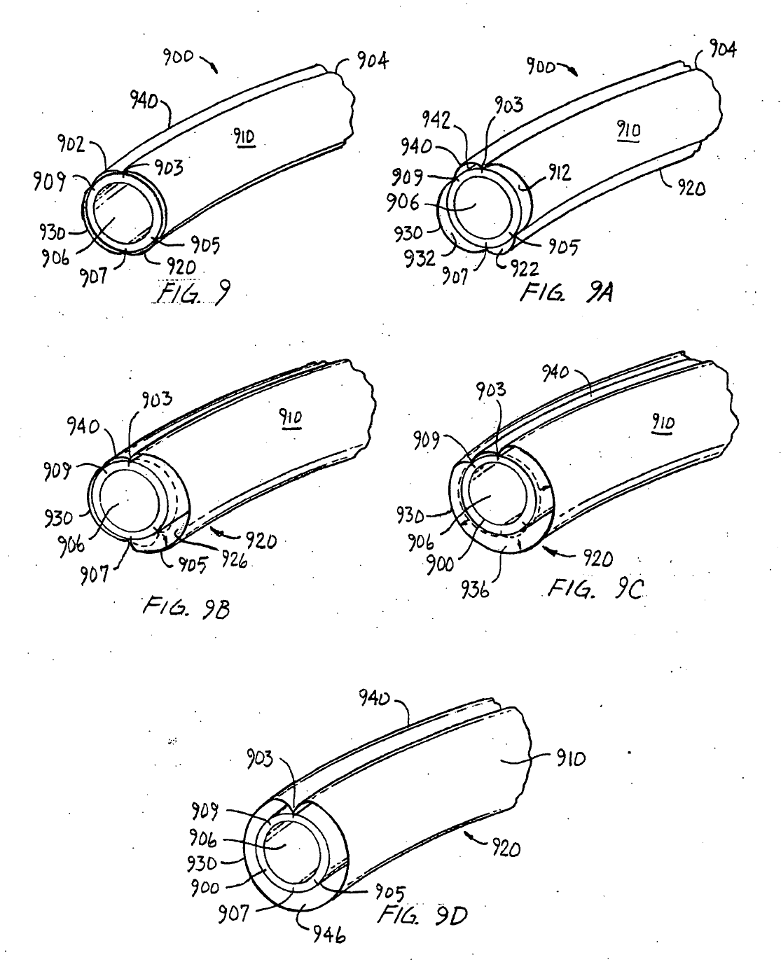

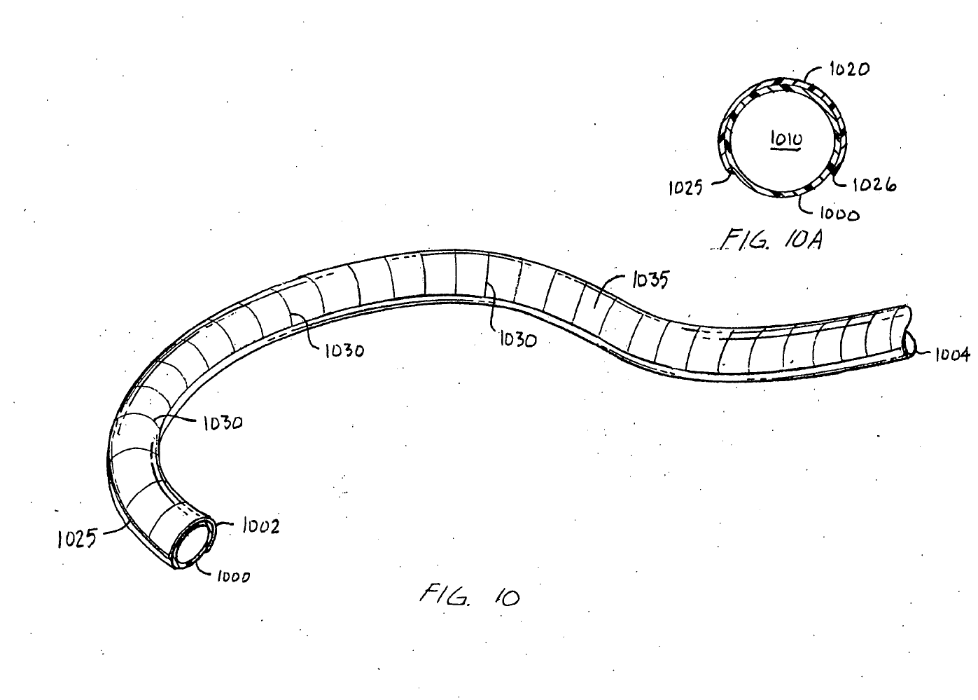

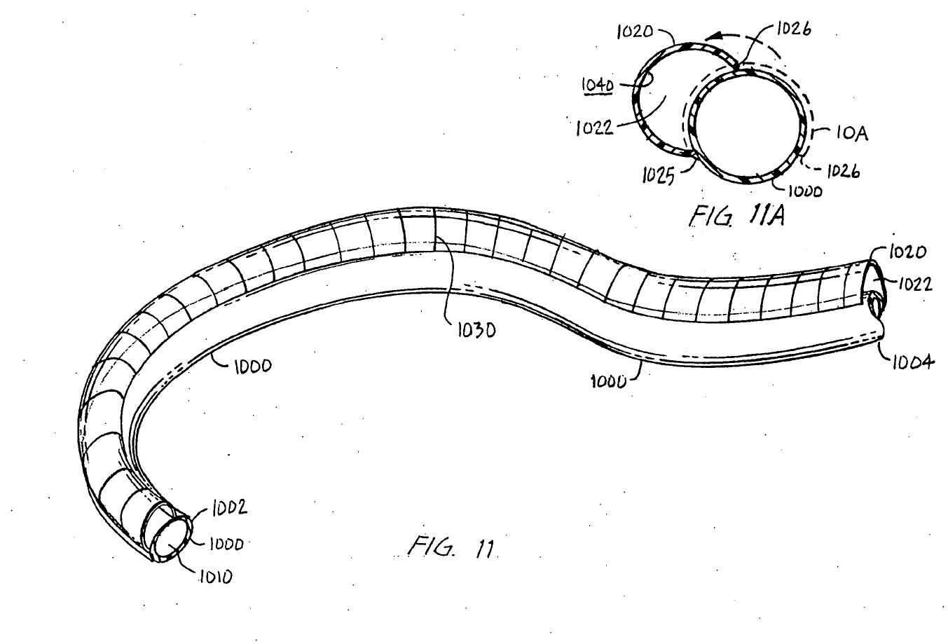

An alternative controlled release embodiment is illustrated in FIGS. 9-9D. The instrument 900 has an elongate body, a proximal end 902, a distal end 904 and lumen 906 therebetween. Four controlled release working channels 910, 920, 930 and 940 are provided. In FIG. 9 the four working channels are shown in a stowed configuration. The channel 910 extends between attachment points 903, 905. The channel 920 extends between attachment points 905, 907. The channel 930 extends between attachment points 907, 909. The channel 940 extends between attachment points 909, 903. Each channel can be releasably attached to and separately deployed from the instrument 900 using any of the deployment techniques described herein or known in the art. As such, there are embodiments of the instrument 900 where, for example, the channels 910, 930 are released into a deployed configuration providing two additional working channels while the channels 920, 940 remain in a stowed configuration. In yet another alternative embodiment, the channels 920, 940 may remain in a stowed configuration but be locally expandable working channel embodiments as described below in FIGS. 14A-C. Still other additional alternative configurations are possible.

In another alternative embodiment, the individual channels 910, 920, 930 and 940 may be separately released and deployed but joined together to form a controlled release, variable size working channel as illustrated in FIGS. 9B-9D. Channel 910 is deployed and then enlarged by deploying channel 920 and releasing attachment point 905 to form lumen 926 (FIG. 9B). The lumen 926 could then be increased by deploying channel 930 and releasing attachment 907 to form working channel lumen 936 (FIG. 9C). Finally, if a single large working channel is desired, then channel 940 could be deployed and the attachment 909 released to form a working channel lumen 946 that is attached to the instrument 900 at attachment 903.

One advantage of the controlled release embodiments is that a smaller channel is deployed and used to pass instruments and perform a procedure while the larger area working channel lumen could be used for irrigation, evacuation or tissue removal and the like. For example, consider the instrument 900 configuration illustrated in the embodiment of FIG. 9C. One advantageous configuration provides for the utilization of a deployed channel 940 for a tool or working conduit to introduce an instrument for a procedure such as the removal of tissue. The tissue removed by the tool in channel 940 would be removed via the larger working channel lumen 936. The lumen 936 provides a larger working channel for irrigation, tissue or material removal or other purposes better accommodated by a larger working channel. Other working channel combinations are also possible. For example, it may be advantageous to have two separate working channels sized for instruments and one other larger working channel. Consider for example the embodiment of FIG. 9A where channels 930, 940 are deployed to form two discrete instrument working channels with lumens 932, 942 respectively. Channels 910, 920 are also deployed with attachment 905 released to form working lumen 926 as shown in FIG. 9B. It is to be appreciated that each of the working channels described in FIGS. 6A-9D may be separated from the instrument and used as a stand alone working channel. Alternatively, a working channel may be separated from the instrument after use and removed from the body while the instrument and other working channels remain in place.

FIGS. 10-11A illustrate an instrument 1000 having an elongate body, a proximal end 1002, a distal end 1004 and a lumen 1010 therebetween. The external working channel on instrument 1000 is provided using a semi-tube 1020. The semi-tube 1020 has an arcuate shape that is not closed and an interior surface 1040. The end view section view of FIG. 10A shows how the semi-tube 1020 conforms to the exterior shape of the instrument 1000 and maintains a low profile in the stowed configuration. A plurality of frame elements 1030 extend along the length of the semi-tube 1020 and are enclosed by cover or sheath 1035 (FIGS. 10 and 11). The frame elements 1030 are flexible structural elements that provide shape to the semi-tube structure. The frame elements may be formed from any suitable metal or plastic and sized depending upon the semi-tube application and dimensions. The sheath 1035 may be made from polymers, e.g., polyethylene and copolymers thereof, polyethylene terephthalate or copolymers thereof, nylon, silicone, polyurethanes, fluoropolymers, poly (vinylchloride), and combinations thereof. The semi-tube 1020 has a flexure point 1025 attached in at least one location to the outer surface of the instrument 1000 and a moveable end 1026. In one aspect, the flexure point 1025 is a continuous attachment between the semi-tube 1020 and the instrument 1000 extending along the length of the semi-tube 1020. In another aspect, the flexure 1025 is discontinuous series of connections between the semi-tube 1020 and the instrument 1000. The semi-tube 1020 extends along the outside of the instrument 1000 and has a stowed configuration against the instrument (FIG. 10A) and a deployed configuration to form a working channel 1022 (FIG. 11A). The interior surface 1040 is against or adjacent the outer instrument 1000 surface when the semi-tube is in the stowed configuration. The working channel formed by a deployed semi-tube is defined by the interior surface 1040 and the surface of the instrument 1000 between the flexure 1025 and the moveable end 1026.

In one embodiment, the frame elements 1030 are flexible and biased towards the deployed configuration (FIG. 11A) but held in place by a suitable restraint. When the restrain is released, the semi-tube 1020 would partially rotate or flex about the flexure point 1025 into the deployed configuration (FIG. 11A) using the return force stored in the frame elements 1030. In one alternative embodiment, the frame elements 1030 are shape memory alloy elements. The shape memory frame elements could be adapted such as by using complementary pairs of SMA frame elements or separately controllable return force elements to transition the semi-tube between the stowed and deployed configurations. In yet another alternative embodiment, the sheath 1035 may be completely or partially replaced or augmented by an electroactive polymer (EAP) sheet that when activated transitions the semi-tube between the stowed and deployed positions. In yet another embodiment, the EAP covering may be used in combination with SMA based frame elements. In yet another embodiment the frame elements 1030 are complementary pairs of SMA elements. In this embodiment, when one part of the complementary pair is activated (i.e., contracts) the semi-tube 1020 is pulled into the deployed condition while at the same time extending the other SMA element in the complementary pair. To transition the semi-tube back into a stowed configuration, the extended SMA element is activated and contracts, pulling the semi-tube from the deployed to the stowed configuration while also extending the other SMA elements.

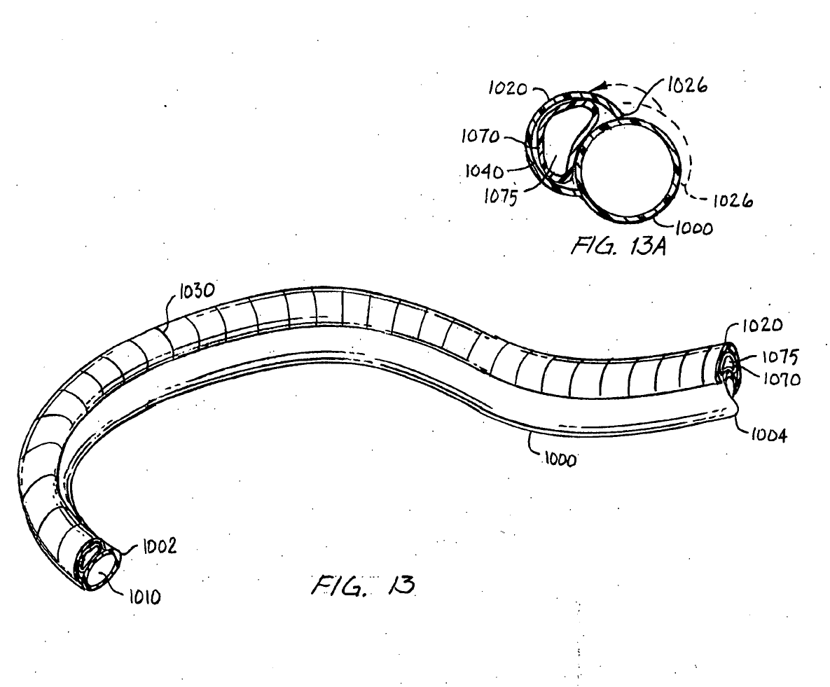

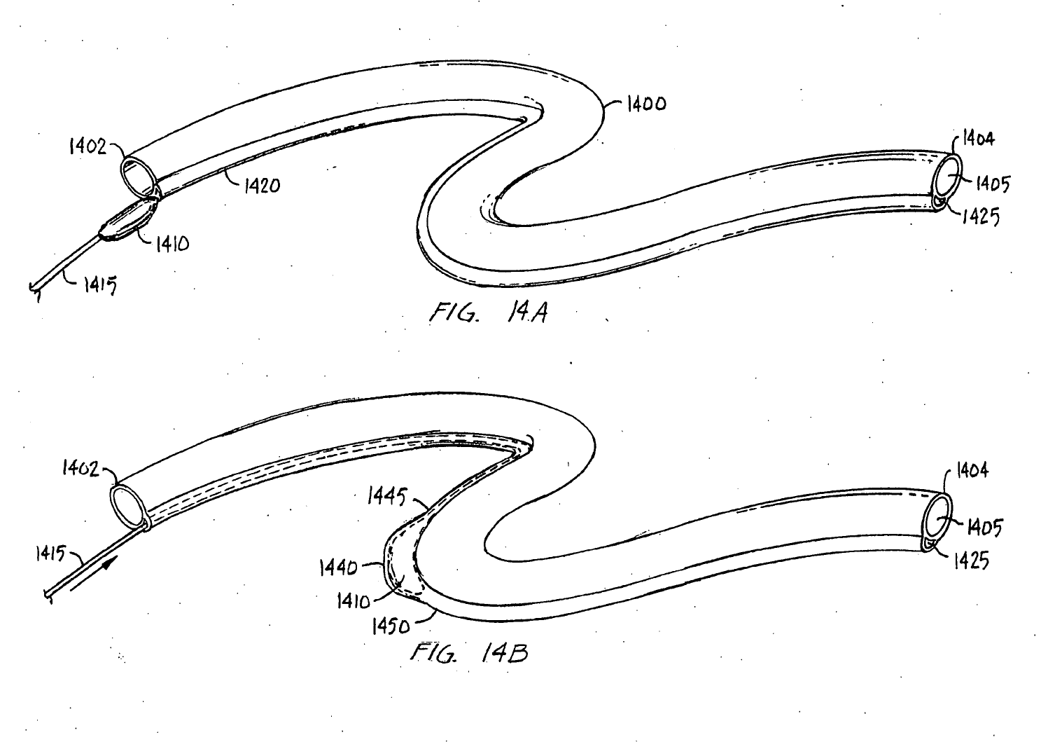

FIGS. 12-13A illustrate an alternative embodiment of the semi-tube external working channel of FIGS. 10-11A. The semi-tube 1020 includes an expandable lumen 1070 disposed between the semi-tube interior surface 1040 and the exterior of instrument 1000. The expandable lumen 1070 may be attached to either the interior semi-tube surface 1040 or the exterior of the instrument 1000. When the semi-tube 1020 is in the stowed configuration, the expandable lumen 1070 is collapsed between the semi-tube interior wall 1040 and the exterior wall of the instrument 1000. FIG. 12A illustrates a stowed semi-tube 1020 configuration and how the semi-tube 1020 and collapsed lumen 1070 conform to and maintain a low profile shape against the instrument 1000. FIGS. 13 and 13A illustrate the semi-tube 1020 in deployed configuration away from the instrument and deployment of the expandable lumen 1070 to form a closed working channel lumen 1075. In one embodiment, the expandable channel 1070 is inflated to form the closed working channel 1075 with a force sufficient to maintain the integrity of the closed working channel 1075 and also maintain the semi-tube 1020 in a deployed configuration. In other words, the semi-tube 1020 is biased into a stowed configuration. When the working channel 1070 is deployed, the expansion of the channel 1070 overcomes the semi-tube 1020 bias and the semi-tube 1020 transitions into a deployed configuration (FIG. 13A). In one specific embodiment, the frame elements 1030 are biased into the stowed configuration (FIG. 12A). When the deployed configuration is desired, the expandable lumen 1070 is deployed, for example, by inflating the interior 1075 or a hollow sidewall of the expandable channel 1070 thereby overcoming the frame member bias and urging the semi-tube 1020 into a deployed configuration (FIG. 13A). When the stowed configuration is desired, the pressure applied to the lumen 1075 or hollow sidewall (not shown, but within the wall thickness of the expandable channel 1070) is reduced or removed, and the frame element 1030 bias returns the semi-tube 1020 to the stowed configuration (FIG. 12A). The semi-tube 1020 and expandable channel 1070 may also be used in combination with SMA and EAP components and/or functionality as described herein.

In another alternative embodiment, the expandable working channel is provided exterior to an instrument using an external working channel having locally expandable dimensions. In contrast to some of the earlier described working channel embodiments, the expandable working channel 1420 in this embodiment may be locally expanded to accommodate the shape of an instrument 1410 advanced using guide 1415 (FIGS. 14A-14C). Rather than a fixed, predetermined channel shape as in some earlier described channel embodiments, the expandable working channel has an original shape (i.e., the unexpanded shape of channel 1020 and lumen 1025) as in FIG. 14A and a deformed shape (FIG. 14B). The instrument 1400 has an elongate body, a proximal end 1402, a distal end 1404 and a lumen 1405 therebetween. The locally deformable channel 1420 extends along the length of the instrument 1400 from the proximal end 1402 to the distal end 1404. The locally deformable channel 1420 has elastic properties that allow for temporary, localized deformation to allow an instrument 1410, for example, to move within lumen 1425. After the instrument 1410 passes, the deformable channel 1020 returns to its original shape (FIG. 14A). FIG. 14A illustrates an instrument 1410 just prior to introduction into the proximal end of the locally expandable working channel 1020. As the instrument 1410 advances distally the working channel 1420 and lumen 1425 deform locally to allow the instrument 1440 to pass. As shown in FIG. 14B the channel 1420 retains its initial diameter in both the proximal and distal ends and in positions immediately proximal 1445 and distal 1450 to the instrument 1410. However, directly adjacent to the instrument 1440 the channel 1420 and lumen 1425 have a locally expanded form 1440 that conforms at least in part to the outer dimensions of the instrument 1410. FIG. 14C illustrates the expandable channel 1420 returning to the original dimensions in the proximal sections where the instrument 1410 has passed and only maintains the locally expanded dimensions 1440 in the area adjacent the instrument 1410.

FIG. 14D illustrates another embodiment of an external working channel that is locally expandable to accommodate an instrument. External working channel 1450 includes a plurality of expandable rings 1455 with a sheath 1460 extending therebetween. Each expandable ring 1455 comprises at least one semi-rigid section 1465 and at least one expandable section 1470 defining a lumen 1480. The expandable working channel 1450 is similar to the expanded working channel 1420 with the added structural benefit of incorporating a semi-rigid section or sections 1465. The semi-rigid section 1465 may be formed from any material capable of retaining its shape with little or only slight deflection when the expandable section 1470 expands. For example, flexible metals or plastics may be used.

The semi-rigid section or sections 1465 are used to maintain a general shape of the external channel 1450 and lumen 1480. The expandable section or sections 1470 along with the expandable sheath 1460 cooperatively flex to accommodate a tool, an instrument or a device transiting through the lumen 1480. Thus, the size and shape of the lumen 1480 is variably adjustable depending upon the number of semi-rigid sections 1465, expandable sections 1470, and the degree of expansion of the expandable sections. In the illustrated example of FIG. 14D there are four semi-rigid sections 1465, 1466, 1467, 1468 spaced between four expandable sections 1470, 1472, 1474, 1476. In this example, the semi-rigid sections 1465, 1466, 1467, 1468 have an arcuate shape to provide a lumen 1480 with a generally circular shape. Other configurations are possible, and more or fewer semi-rigid sections and expandable sections may be provided. For example, there may be only one semi-rigid section 1465 and one expandable section 1470 used to form a closed shape defining the lumen 1480.

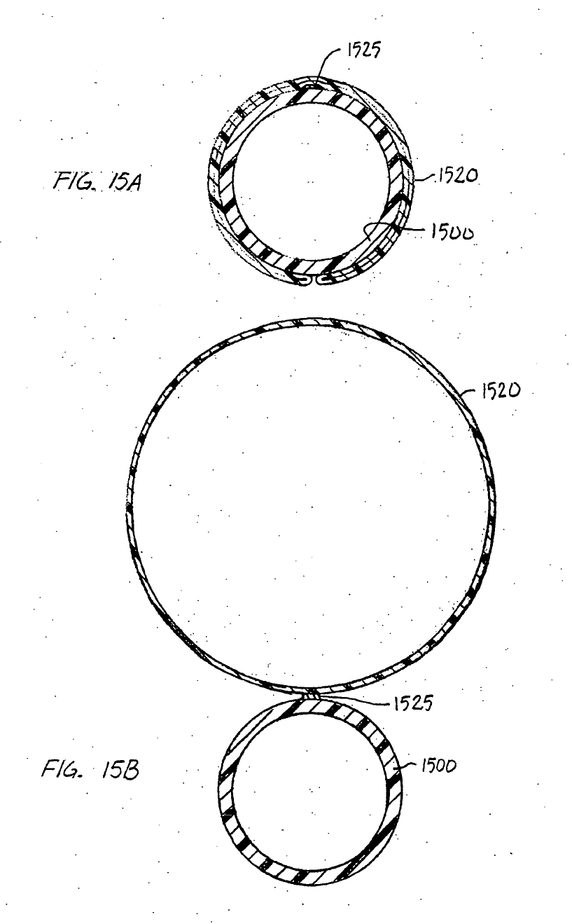

Many of the illustrative external working channel embodiments described herein are smaller than or about the same size as the attached instrument. However, it is to be appreciated that the external working channel may also be larger than the attached instrument. FIGS. 15A and 15B illustrate one illustrative embodiment of this concept. A working channel 1520 is illustrated in a stowed configuration about an instrument 1500 (FIG. 15A). The working channel 1520 is attached to the instrument 1500 along attachment 1525. Attachment 1525 could be a continuous attachment along the length of the instrument or a series of attachment points between the instrument 1500 and working channel 1520. When the working channel 1520 is in a deployed configuration, the working channel 1520 is larger than the instrument 1500 (FIG. 15B). In conventional instruments, an increased size internal working channel may be provided, but increasing the size of the working channel also substantially increases the size of the instrument delivering the working channel. As is clear from FIGS. 15A, 15B, expandable, external working channels of the present invention can provide larger working channels—even working channels larger than the instrument itself—without a substantial increase in instrument size. Moreover, unlike conventional internal working channels and instrument having fixed dimensions, working channel embodiments of the invention may also be fully deployed or partially deployed to provide a range of working channel lumen sizes. In other words, the working channels of the present invention are not confined to only stowed and deployed configurations. Intermediate deployment configurations are also possible. As such, there are working channel embodiments where a single external expandable working channel may provide a wide range of working channel lumen sizes depending upon the degree of working channel deployment.

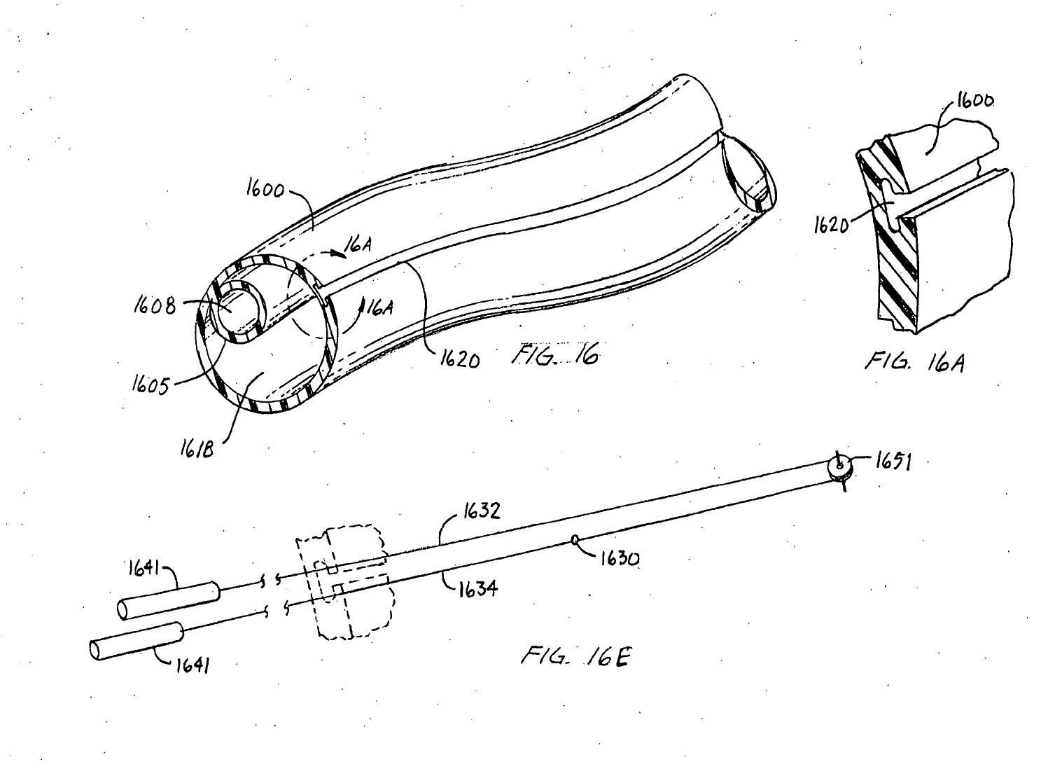

FIG. 16 illustrates a controllable instrument 1600. Controllable instrument 1600 has only a visualization channel 1608 shown within the lumen 1618. For clarity, other auxiliary components or channels such as an irrigation channel to rinse a lens used with the visualization channel 1608 or controls to steer the instrument 1600 are omitted. However, the controllable instrument 1600 does not have a working channel within lumen 1618. Earlier described controllable instrument embodiments include an attached external working channel. As such, the external working channel is selected in advance. In contrast, the steerable instrument 1600 does not have an attached working channel but instead has at least one guide 1620 to receive a working channel. In this way, the controllable instrument 1600 may be initially used as an inspection device. Thereafter, if the inspection reveals a condition in need of treatment or further examination, then an external working channel may be provided using the guide 1620. Rather than insert an instrument with a pre-determined external working channel size, the instrument has no external working channel and selects one only if needed and/or based on size requirements of a procedure to be performed. In the illustrated embodiment, the guide 1620 extends the length of the controllable instrument 1600. In alternative embodiments, the guide 1620 or one or more guides 1620 may extend to a selected length or depth along the instrument 1600 (see, e.g., FIG. 21).

As best seen in FIG. 16A, the guide 1620 is a T-shaped channel formed in the controllable instrument sidewall. Other guide shapes are possible. In one alternative embodiment, the guide is a closed shape. In still another embodiment, the closed shape guide may be coupled to a pressure source so that a carrier adapted to translate within the closed shape guide may be moved through the closed shaped guide using differential pressure applied to the closed shape guide. In another alternative embodiment, the guide is a rail above the instrument sidewall rather than a channel within the sidewall. FIG. 16B illustrates an embodiment of a steerable instrument 1600 having a T-shaped rail guide 1690.

FIG. 16C illustrates an exemplary carrier 1630. The carrier 1630 is used to translate working channels, instruments or other items along the guide. In the illustrated embodiment, the carrier 1630 is sized and shaped to fit within and translate along the guide 1620. Likewise, a carrier adapted for use with the guide rail 1690 would be adapted to receive the guide rail 1690 (FIG. 16B). Accordingly, a carrier is adapted to engage and translate along a guide. In addition, the carrier is configured to receive an external working channel, an instrument, or other item to be translated along the steerable instrument guide. A connection point 1640 is provided to couple an item to the carrier 1630. FIG. 16D illustrates a guide 1631 with an instrument 1670 attached via connection point 1640. In this embodiment, straps 1642 are used to keep the instrument 1670 in place on the connection point 1640. The connection point 1640 and the instrument 1670 may be coupled together using any suitable attachment method. Additionally, the instrument and/or the carrier may be equipped with a release to allow the instrument to be separated from the carrier.

Carrier translation along a guide may be accomplished in a number of ways. In the case of carrier 1630, cables 1632, 1634 are used for proximal and distal translation, respectively (FIG. 16C). Cable 1632 is attached to the carrier 1630 via attachment point 1636. Cable 1634 is also attached to carrier 1630 using an attachment point (not shown). The cables 1630, 1634 advantageously allow the carrier 1630 to be pulled along the guide 1620 in either direction. In one alternative embodiment, the cables may be part of a pulley arrangement as illustrated in FIG. 16E. In this embodiment, handles 1641 are connected to cables 1632, 1634 and are used in conjunction with pulley arrangement 1651 attached to the steerable instrument. Pulling one of the handles 1641 will translate carrier 1630 along the steerable instrument guide. In FIG. 16D, carrier 1631 illustrates the use of cable pass throughs 1647, 1649 for cables 1632, 1634.

Some external working channel embodiments may also have atraumatic tips or distal portions adapted to deflect tissue as the external working channel advances. The external working channel may include an inflatable structure such as a balloon. The atraumatic tip may be virtually any shape that would help prevent pinching, tearing adjacent tissue as the external working channel advances.

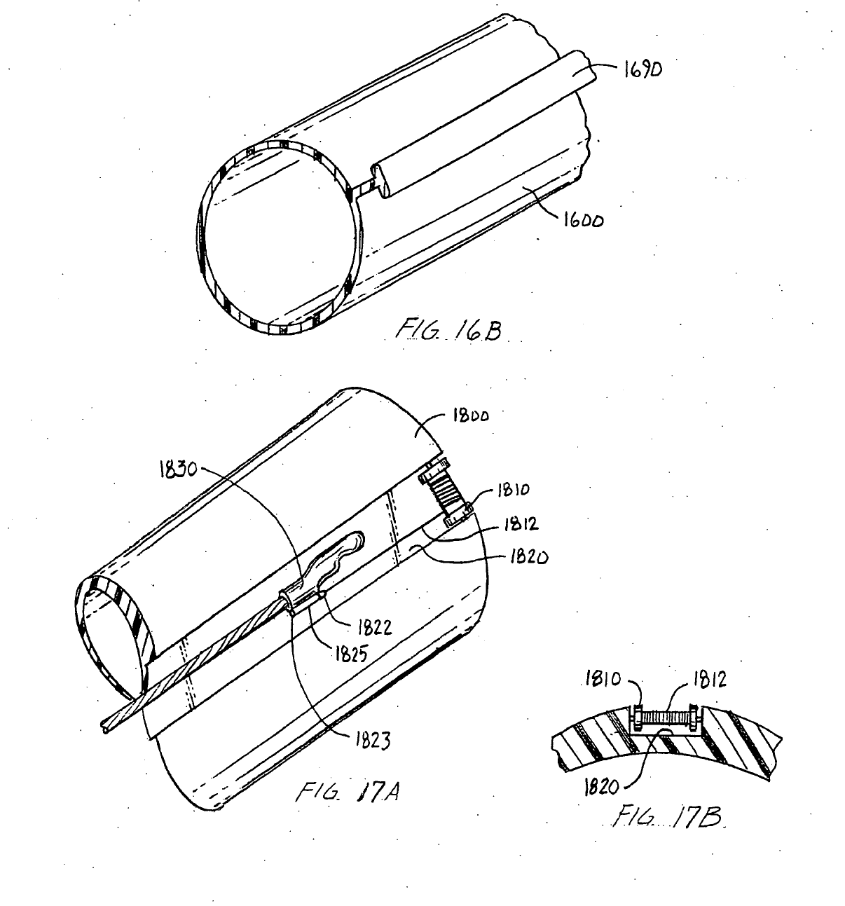

A motorized spool 1810 may be placed distally on the instrument 1800 as an alternative to the pulley arrangement 1651 (FIGS. 17A and 17B). The spool 1810 is arranged within guide channel 1820 in the illustrative embodiment. The spool 1810 is used to draw up cable 1812 (FIG. 17A). A carrier 1825 may be connected to an instrument 1630 or other object such as an expandable working channel, for translation along the controllable instrument 1800. The carrier 1825 is attached to cable 1812 at distal attachment point 1822. A cable (not shown) may also be attached to proximal attachment point 1823 to withdraw the carrier 1825 with or without the instrument 1830. The use of the cable attached to attachment point 1823 allows for spool 1810 to advance the carrier 1825 distally while the cable attached to point 1823 could be used to proximally withdraw the carrier 1825.

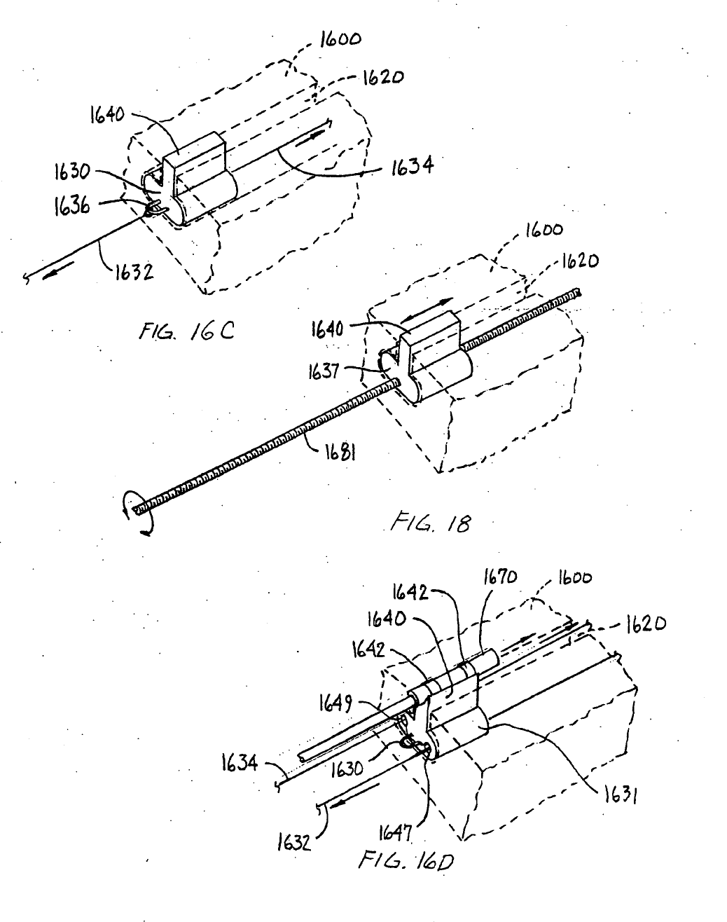

In another alternative embodiment, a lead screw is used to advance a carrier along a guide (FIG. 18). In the illustrative embodiment, the lead screw 1681 is positioned along the guide 1620. A carrier 1637 is adapted to engage with the lead screw 1681. When the lead screw 1681 rotates, the carrier 1637 moves along the guide 1620 as indicated by the arrows.

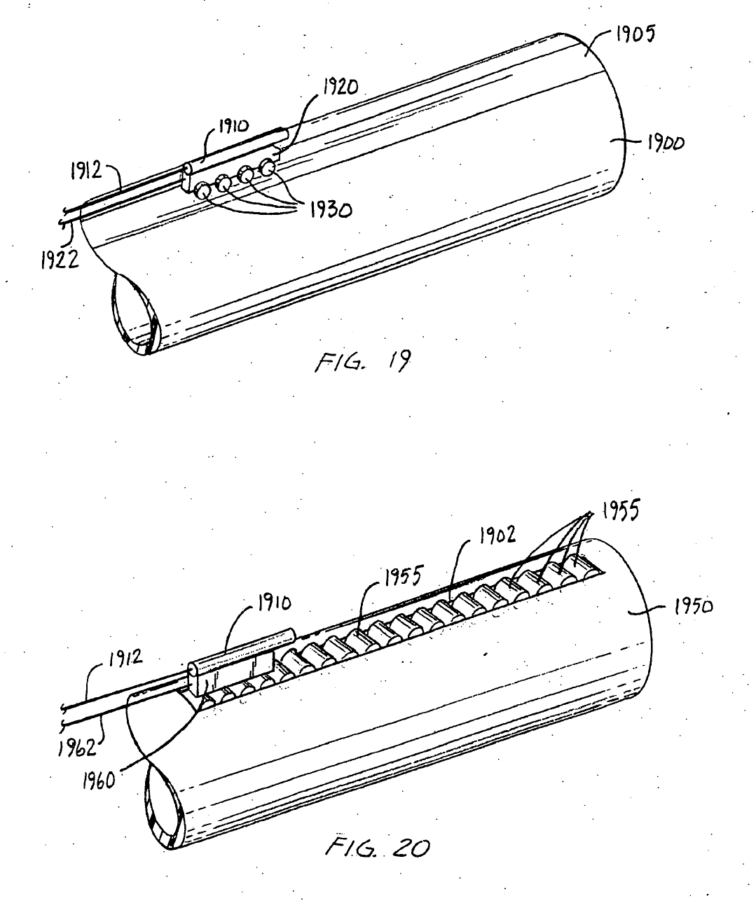

FIGS. 19 and 20 illustrate additional alternative guide embodiments. In FIG. 19, a magnetic guide strip 1905 extends along the controllable instrument 1900. A carrier 1920 has metallic rollers or wheels 1930 that are attached to and follow along the magnetic guide strip 1905. A push rod 1922 is attached to carrier 1920 to move the carrier 1920 along the guide strip 1905. Alternatively, the earlier described pulley or spool devices may be used to move the carrier 1920. In yet another alternative embodiment, the carrier 1920 is motorized and self propels itself along the magnetic guide strip 1905. In additional alternative embodiments, both the rollers 1930 and strip 1905 are magnetic or the rollers 1930 are magnetic and the strip 1905 is a metallic material.

FIG. 20 illustrates another alternative guide embodiment. A plurality of rollers 1955 are arrayed along the controllable instrument 1950 to form a roller guide 1902. A carrier 1960 has a magnetic face (not shown) that is attracted to and rides along the rollers 1955. As before, other roller 1955/carrier 1960 combinations are possible. For example, one or both of the roller 1955/carrier 1960 may be magnetic or otherwise configured to use magnetism or other connection forces to retain the carrier 1960 on the rollers 1955.

It is to be appreciated that while the previously described illustrative embodiments detail the operation of a single guide, more than one guide may be provided and used. Consider the embodiment of the controllable instrument 2100 in FIG. 21. The controllable instrument 2100 has three guides 2180 distributed about the instrument 2100. More or fewer guides 2180 may also be used. The guides 2180 may have any shape and configuration such as those described herein or others. Each of the guides 2180 may be used individually or two or more guides may be used cooperatively. The multiple guide arrangement allows for more than one instrument or external channel or other items to be run in along the guide 2180. The instrument 2100 also illustrates the internal channels 2170, 2172 and 2174 used, for example, to provide illumination, visualization, irrigation, suction and other auxiliary functions in support of operating and controlling the instrument 2100. The controllable instrument 2100 does not, however, have an internal working channel.

While described in terms of use with an instrument, it is to be appreciated that the techniques and devices described with regard to FIGS. 16-21 are applicable to the movement of devices through and within an external working channel. As such, other external working channel embodiments may include one or more features described in FIGS. 16-21.

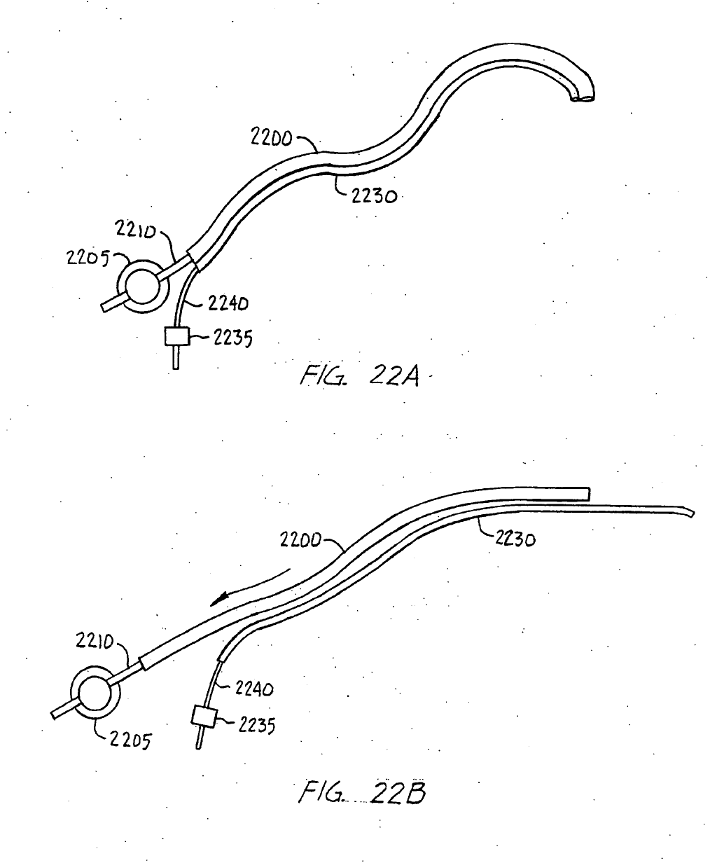

In still other alternative embodiments, the external working channel may be independently controllable from the controllable instrument. Consider the illustrative embodiment of FIG. 22A. The controllable instrument 2200 includes a handle 2205 and control umbilical 2210 connecting the handle 2205 to the controllable instrument 2200. An external working channel 2230 is attached to and extending the length of the controllable instrument 2100. The external working channel 2230 is shown in the deployed configuration. The external working channel 2230 may also be attached to the steerable instrument 2200 and configured in a stowed configuration as discussed above. Like the controllable instrument 2200, the external working channel 2230 also has a handle 2235 connected to a control umbilical 2240. In one embodiment, the external working channel 2230 is a functioning steerable instrument with the same features and characteristics as the steerable instrument 2200. For example, the working channel 2230 may include visualization, illumination or imaging capabilities. As best seen in FIG. 22B, when the external working channel 2230 is detached from the controllable instrument 2200 the controllable instrument 2200 may be withdrawn leaving the controllable external working channel 2230 in place and operable. Any of a variety of conventional attachment and release schemes may be used to join the controllable external working channel 2230 to attach and release it from the steerable instrument 2200. In additional alternative embodiments, the detachable external working channels of the present invention may also be adapted for delivery of tools and other instruments as discussed in FIGS. 16-21.

One advantage of the detachable external channel embodiments is that once the controllable instrument 2200 has been used to deliver the external channel 2230 into the desired position within the body and detached, the controllable instrument 2200 can be removed thereby providing additional space for performing procedures using the external channel. In one embodiment, the external channel 2230 remains stowed until the controllable instrument 2200 is withdrawn. Once the controllable instrument 2200 is withdrawn, the external channel 2230 transitions to a deployed configuration. Alternatively, the external channel 2230 may gradually transition to a deployed configuration as the controllable instrument is withdrawn or may transition to a deployed configuration all at once after removal of the controllable instrument 2200. In another alternative embodiment, the external channel 2230 is positioned by the controllable instrument 2200 in a desired location within the body. Thereafter, the external channel 2230 transitions to a deployed configuration and is used as a working channel to provide access within the body in proximity to the desired location. Once access is no longer required, the handle 2235 and cables 2240 are used to with draw the external channel 2230.

FIG. 23 illustrates an embodiment of an inspection device 2300. The inspection device 2300 is illustrated in operation within a lumen 2305. The inspection device 2300 has a generally rounded conical shape with a distal tip 2302 and a proximal end 2304. The proximal end 2304 is shaped to expand into a sealable relationship with the interior wall of lumen 2305. The proximal end 2304 may include a ring sized and adapted to expand the proximal end into atraumatic contact with the interior wall of lumen 2305. The proximal end 2304 seals with the inner wall of lumen 2305 sufficient to form a fluid or gas barrier to fluids or gases later introduced proximal to the inspection device 2300. The inspection device 2300 is formed from any suitable material that can hold liquid or fluid introduced to move the device through the lumen 2305. The material may also be selected as a biocompatible material or include a coating that does not irritate the interior of lumen 2305. Optionally, the inspection device 2300 may include structural supports or a flexible form in the conical shape that is covered. The use of a structural support or form has the additional advantage of more evenly distributing the applied pressure within the inspection device 2300.

In the illustrated embodiment, two internal channels 2330, 2320 are provided within the inspection device 2300 and connected to the distal end 2302. In one exemplary embodiment, the channels 2320, 2330 cooperate to provide illumination and visualization of the interior of lumen 2305. One or both of the channels 2320, 2330 may be used as a guide for the later delivery of instruments, a working channel or other items within the lumen 2305. In operation, air or other fluid introduced proximally to the inspection device 2300 causes distal movement of the device through the lumen 2305 as indicated by the arrows. Images of the interior of lumen 2305 are provided by the channels 2320, 2330 alone or in combination as is typical in the endoscopic imaging arts. The images may be inspected in real time as the device 2300 advances or may be recorded and later examined. One advantageous operation includes rapidly advancing the inspection device 2300 through the lumen

Optionally, the illustrative embodiment shows an embodiment having a guide wire 2312 attached to the proximal end at attachment point 2314. In this optional embodiment, the guide wire 2312 trails behind the device 2300 thereby providing a separate guide for subsequent delivery of additional devices or instruments.

In another alternative embodiment, the endoscope 100 described above is modified to have one or more guides. In addition, the endoscope 100 has been modified to remove working channels within the endoscope 100. In an alternative embodiment, the endoscope 100 is a pediatric endoscope with any internal working channel(s) removed and adapted to have one or more guides. In an exemplary operation, an embodiment of the endoscope 100 is advanced through the colon of a patient. While advancing, the endoscope captures images of the colon interior, allows for real time examination and position marking, records endoscope controller commands, and creates a map of the colon just to name a few of the functions. Additional details of the operation and functionality of embodiments of the endoscope 100 are further described in U.S. Pat. No. 6,468,203. Moreover, each of the functions and capabilities described above may also include an indication of axial position along the scope, in the colon and/or on the created map.

In one exemplary example, the endoscope 100 embodiment has also been adapted to include 4 guides arranged about the perimeter of the endoscope. Similar to the illustrative embodiment in FIG. 21, the guides are evenly spaced and positioned at the 12 o'clock, 3 o'clock, 6 o'clock, and 9 o'clock positions. For purposes of discussion, there are three polyps identified during the initial colonoscopy and map creation. The polyps are located within the colon as follows: polyp 1 is at a axial depth of 15 cm at the 3 o'clock position, polyp 2 is at a axial depth of 65 cm⁻¹ at the 6 o'clock position and polyp 3 is at a axial depth of 128 cm at the 10 o'clock position. These locations are merely examples and any of a number of location terminology or descriptions may be used to identify a location of interest within the colon. The endoscope is advanced automatically to a position determined by the generated map. The generated map may have stored within it or related to it additional information related to the condition of the colon, organ or body region into which the endoscope will be directed under the control of the motion controller. The additional information may come from other imaging modalities provided in real time to assist in directing the endoscope to the desired position for performing a surgical, therapeutic and/or diagnostic procedure. Once the endoscope is positioned where desired, the external working channel is detached, and the endoscope removed.

In one specific embodiment, the polyp locations are stored in electronic memory and related to the electronically generated map of the colon. In one illustrative method to remove the polyps, the endoscope 100 is advanced beyond the furthest polyp (i.e., a depth of 128 cm). Next, depending upon the size of working channel desired, an external working channel is attached to a suitable carrier and introduced into one of the guides. In this example, polyp 3 is at a depth of 128 cm at the 10 o'clock position so either the 12 o'clock or 9 o'clock guide is a good choice. Next, the carrier is introduced into the guide and, under control of the electronic controller, advanced to a depth of 128 cm. Thereafter, with or without the endoscope 100 in place, the channel is deployed to form a working channel for the removal of polyp 3. After this polypectomy is completed, the working channel may be detached from the carrier and withdrawn using the techniques described herein or the carrier may be removed with the working channel attached. In a similar fashion, an external channel is delivered using the guide at 3 o'clock to remove polyp 1 and an external channel is delivered using the guide at 6 o'clock to remove polyp 2. In this fashion the endoscope is advanced to access the furthest distal polyp and then as it is withdrawn proximally, each next most distal polyp is removed.

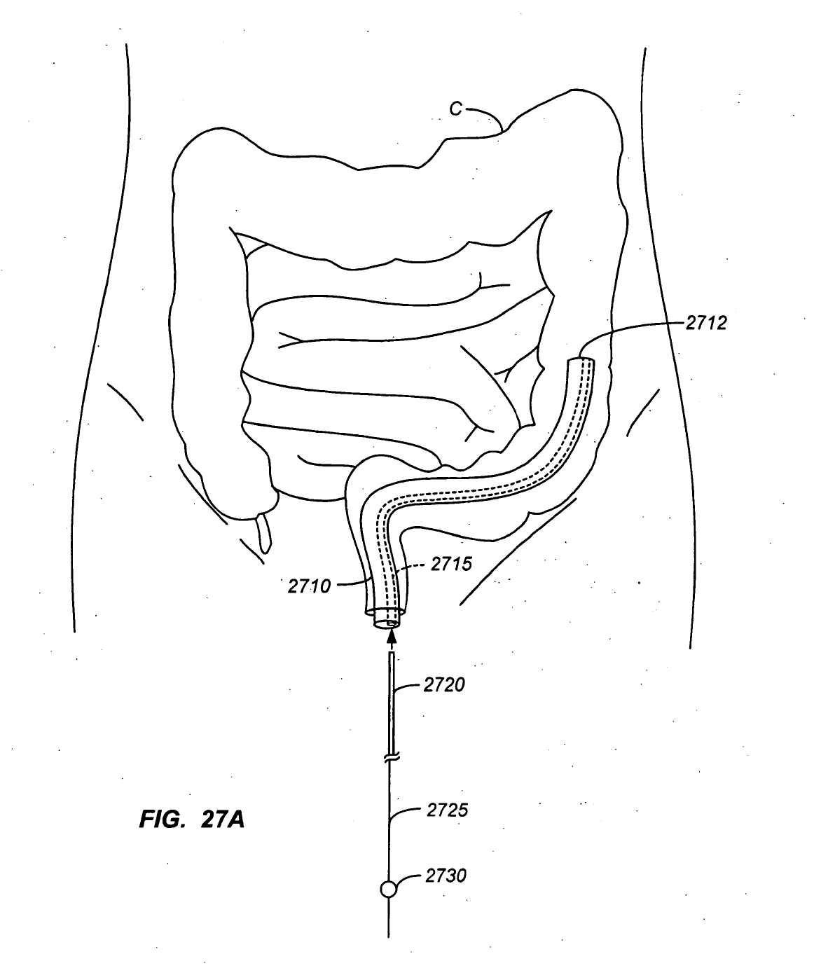

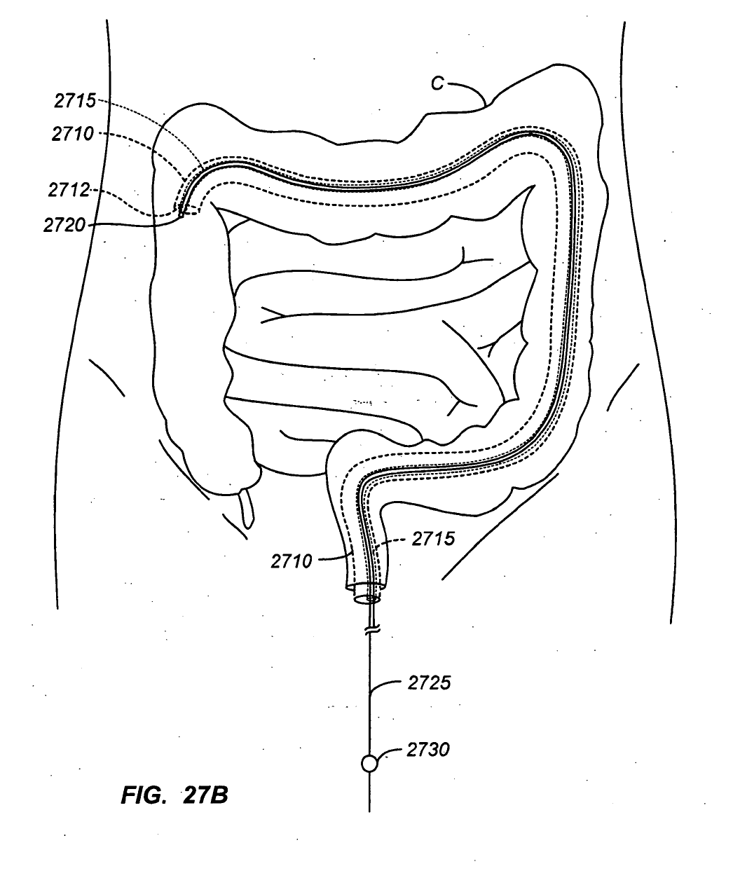

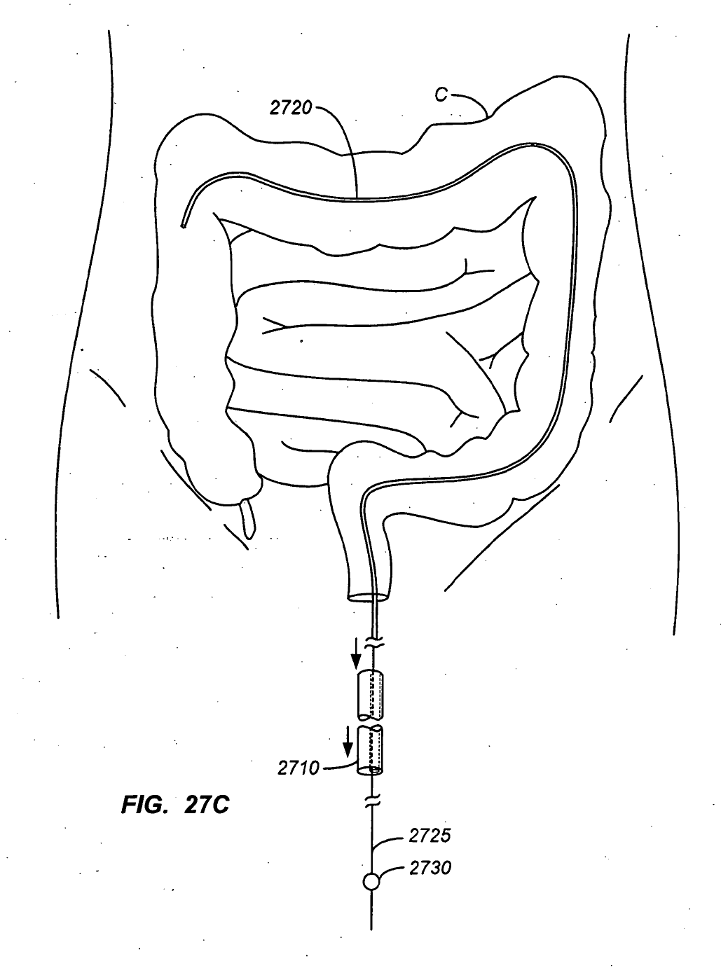

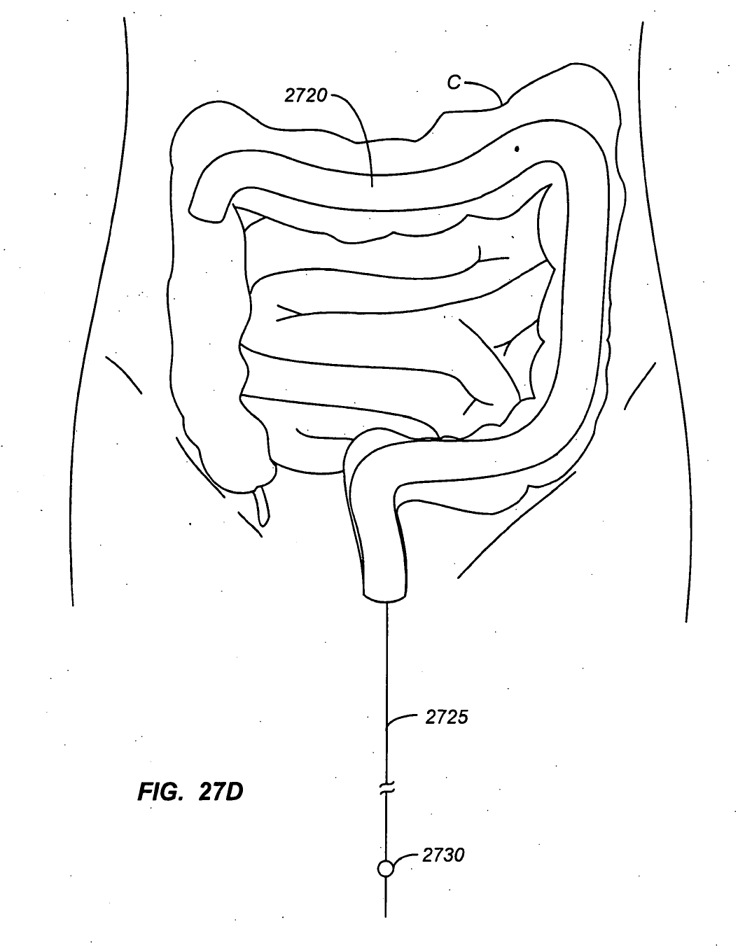

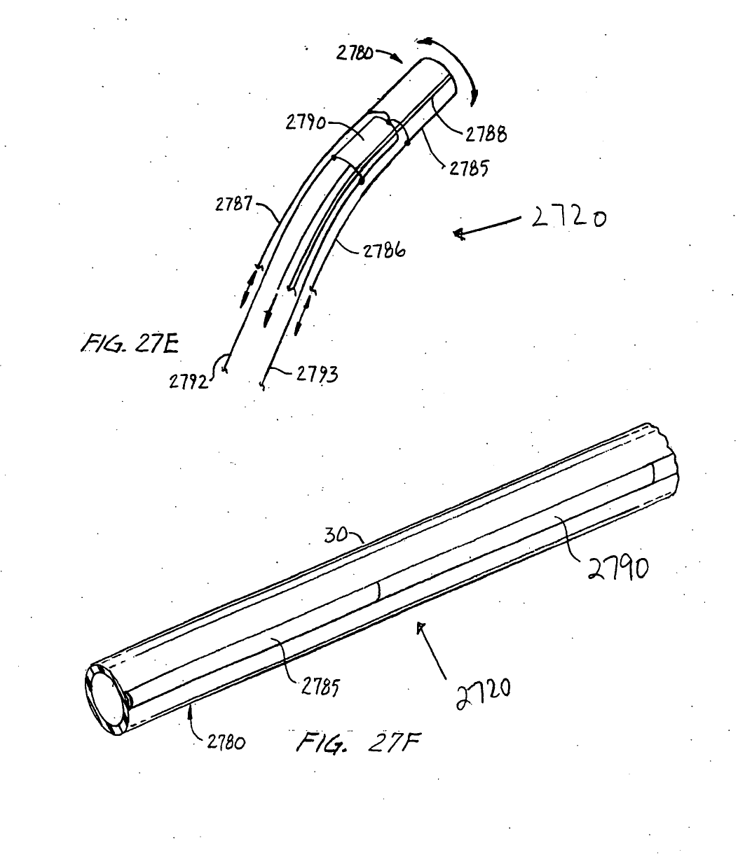

In an alternative embodiment, the working channel of a conventional endoscope may be used to deliver an external working channel according to the present invention. In this embodiment, a conventional endoscope 2710 will be described delivering an external working channel 2720 to a portion of the colon C. First, the endoscope 2710 is advanced within the colon C (FIG. 27A) to a desired position (FIG. 27B). In general, the endoscope distal end 2712 or exit of the working channel 2715 is positioned distally to correspond to the distal most position of the external working channel 2720. The endoscope may be positioned within the body—in this example within the colon—using conventional techniques. Alternatively, in another aspect, the endoscope 2710 is guided using external imaging modalities and techniques, described herein alone or in combination with the computer controlled steerable segmented endoscope described above and in U.S. Pat. No. 6,468,203 incorporated herein by reference.

Next, an external working channel 2710 is advanced along the working channel 2715 until it exits the distal end 2712 (FIG. 27B). External working channel 2720, when in a stowed configuration (i.e., FIGS. 27A, 27B and 27C), is sized to fit within the working channel dimensions of existing endoscope and controllable instrument working channels. In this embodiment, the working channel 2715 also has controls 2730 connected to the external working channel 2720 using a suitable umbilical connection 2725. Controls 2730 and umbilical 2725 are adapted to the capabilities of the external working channel 2720. For example, if the external working channel 2720 has steering capabilities (for example, left/right and up/down tip control as further described below) and/or visualization capabilities (for example, a fiber optic system for lighting and/or visualization) then the control 2730 and umbilical 2725 are adapted to provide tip steering control and visualization in a manner know to those of ordinary skill in the endoscopy arts.

Next, the endoscope 2710 is withdrawn from the colon leaving the stowed external channel 2720 in place (FIG. 27C). Thereafter, the external channel 2720 is configured into a deployed configuration (FIG. 27D). The deployed configuration of FIG. 27D provides a larger working channel available for performing a procedure or otherwise inspecting the colon than the working channel 2715 provided by endoscope 2710 or otherwise available using the working channel of a conventional endoscope. The delivery and deployments steps are described above may be performed in a different order.

FIG. 27E illustrates an embodiment of an external working channel 2720 having a controllable tip 2780 and a light and/or visualization channel 2788. In the illustrated embodiment, the steerable tip 2780 has two segments—a distal segment 2785 and a proximal segment 2790 that controllably articulate to provide left/right and up/down control of the steerable tip 2780. Movement of the segments is accomplished, for example, using control cables 2786, 2787 for distal segment 2785 and control cables 2792, 2793 for proximal segment 2790. Steerable tip 2780 control using the two segments 2785, 2790 through use of cables 2786, 2787, 2792, and 2793 is performed using conventional control techniques known to those in the endoscopy arts or those control systems and techniques described in U.S. Pat. No. 6,468,203, incorporated herein by reference.

Advantageously, the segments forming the steerable tip may, like the external working channels described herein, be positioned in both stowed and deployed configurations in order to economize space needed during delivery of the working channel on or in the delivery instrument. FIG. 27F illustrates one embodiment of an external working channel having steerable segments where the external working channel including the segments is in a stowed configuration. In this illustrative embodiment, the delivery instrument is an endoscope 30 adapted to carry an external working channel having steerable segments. The endoscope may, alternatively, be configured to carry the external working channel having steerable segments within a working channel in the interior of the endoscope. In the stowed configuration of the illustrative embodiment, the segments 2785, 2790 are collapsed and nearly flat arrangement against the endoscope. This illustrative embodiment shows the steerable external channel exterior to the endoscope. Other configurations are possible. For example, the endoscope may have a working channel in the interior of the endoscope having an arcuate, crescent or other cross section shape configured to receive a steerable external working channel in the stowed configuration.

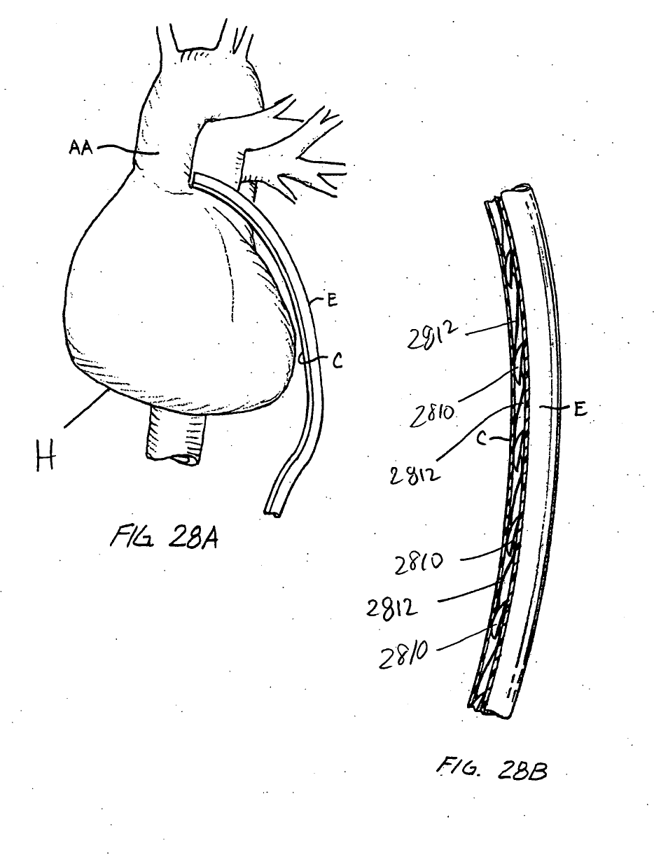

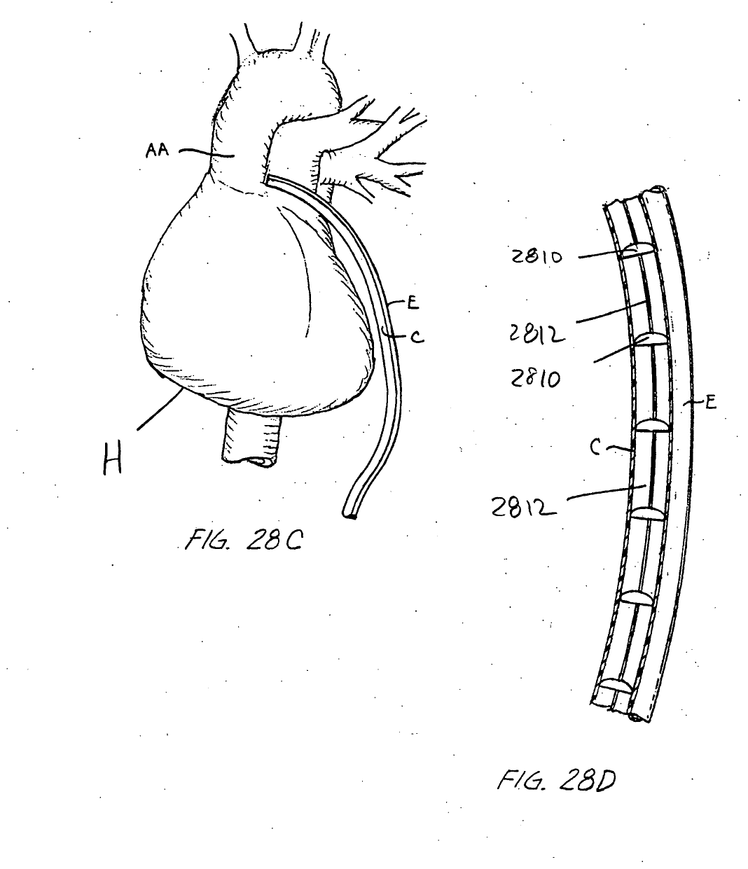

Other embodiments of the external working channel of the present invention may include rigidizable elements or other mechanisms or means for locking the shape, position and/or size of the external working channel. An aspect of this type of external channel will be described with regard to FIGS. 28A-28F.

FIG. 28A illustrates an endoscope E adapted to deliver a working channel C within the body. In this illustrated example, the endoscope E is maneuvered to a position on the heart H adjacent the ascending aorta AA. FIG. 28B is a cross-section view of the endoscope E and channel C of FIG. 28A. The channel C is in a stowed/unlocked position and has a diameter less than the diameter of the endoscope E. In this illustrative embodiment, the channel C has a plurality of rigidizable elements 2810 connected using a cable 2812. In the unlocked position of FIG. 28B, the rigidizable elements 2810 present a reduced profile within the channel C, and there is slack in the cable 2812 between the rigidizable elements 2810. The channel C is releasable couple to the endoscope E using suitable connections that allow the channel C to be delivered by the endoscope E and then detached when desired as discussed below.

Next, as illustrated in FIGS. 28C, 28D, the rigidizable elements are positioned into a locked condition by tensioning the cable 2812 as the channel C transitions from a stowed condition (FIG. 28B) to a deployed position (FIG. 28D). It is to be appreciated that the operation of locking the channel C may occur after or during the transition of the channel C from a stowed to a deployed condition. In other embodiments, the operation used to lock the rigidizable elements or other means used to lock the position of the channel C is also the mechanism or operation used to transition the channel C from a stowed to a deployed configuration. The channel C now provides a rigid working channel from outside the body to a desired position within the body. In the illustrated example of FIG. 28E, the desired position is near the ascending aorta AA.

Once the channel C is positioned and locked where desired, the channel C is detached and/or slideable moveable from the endoscope E (FIG. 28E). As illustrated in FIG. 28F endoscope E may be separately maneuvered to observe and/or assist in a procedure performed using the channel C. In the illustrated embodiment of FIG. 28F, the endoscope E advances distally so that the optic system of endoscope E is used to observe the distal end of channel C and/or use the working channel within the endoscope E to provide additional tools to perform a procedure in conjunction with tools provided via channel C.

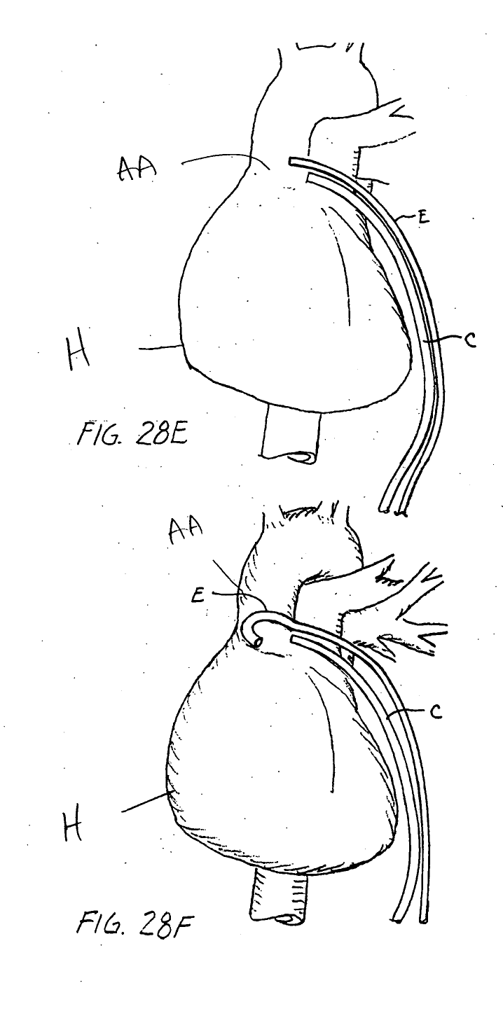

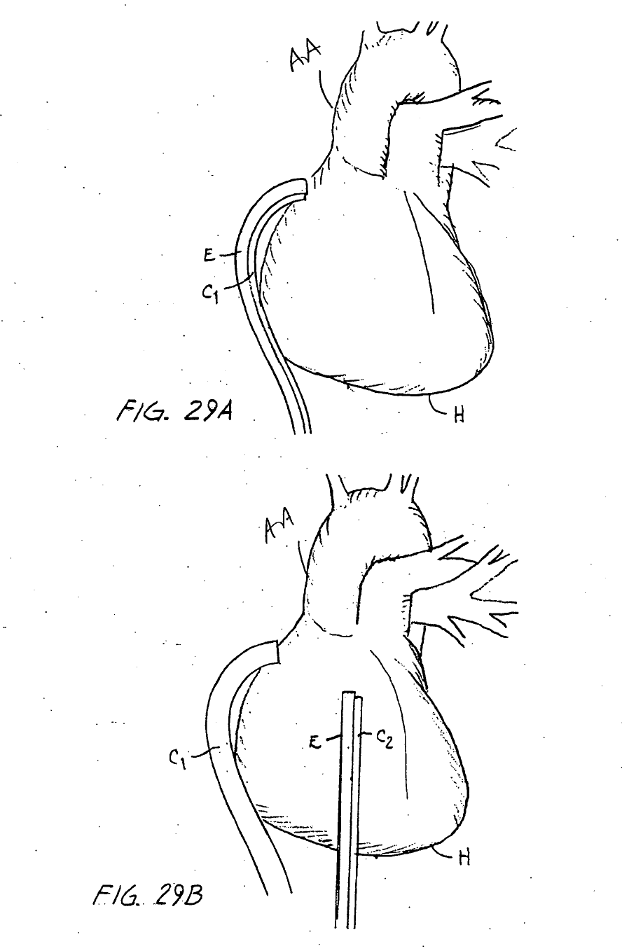

Embodiments of the present invention are not limited to the use of a single external channel C working in cooperation with an endoscope E. Depending upon the specific surgical, therapeutic and/or diagnostic procedure being performed, a plurality of external channels C may be delivered via the endoscope E to non-evasively provide multiple, independent access points to a portion of the body. FIGS. 29A-29D illustrate the delivery and positioning of three working channel C1-channel C3 to a position on the heart H adjacent the ascending aorta AA.

In FIG. 29A, the endoscope maneuvers into the desired position to place the working channel C1. During delivery, the channel C1 advantageously remains in a stowed condition or a condition where the diameter of the channel C1 is less than the diameter of the endoscope E. Once positioned, channel C1 is detached from the endoscope E, transitioned to and is locked in a deployed configuration thereby forming a first working channel to access a region within the body (FIG. 29B). In similar fashion, the second channel C2 is positioned (FIG. 29B) and deployed (FIG. 29C) and the third channel C3 is positioned (FIG. 29C) and deployed (FIG. 29D). FIG. 29D illustrates how working channel embodiments of the present invention may be advantageously delivered and positioned into a portion or region of the body to provide multiple, simultaneous access ports to perform surgical, therapeutic, and/or diagnostic procedures. Moreover, the endoscope E may also be used to observe and/or provide lighting or visualization of the portion or region accessed by the channels C1, C2 and C3.

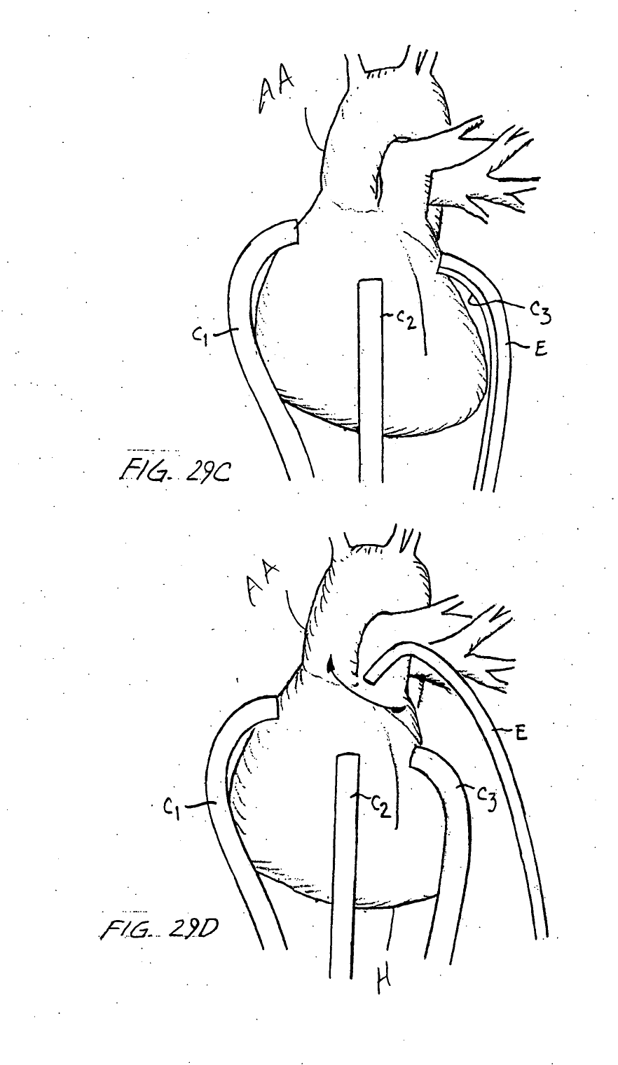

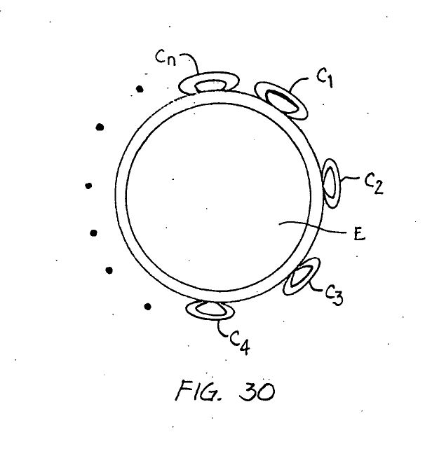

The illustrated embodiments of FIGS. 28A-29B describe an external working channel delivery method where a single external channel C is delivered using an endoscope. The endoscope E may deliver working channels using the endoscope E working channel (i.e., FIGS. 27A-27B), an external delivery mechanism (i.e., FIGS. 16-21) or other techniques for endoscopic delivery known to those of ordinary skill. Alternatively, an endoscope may be adapted to deliver and detach multiple working channels during a single channel delivery process or a continuous channel delivery process. One embodiment of an endoscope adapted to deliver multiple external working channels is illustrated in FIG. 30. The endoscope E has a plurality of external working channels C₁-Cₙ distributed about an exterior surface in the endoscope. Each of the working channels C₁-Cₙ are illustrated in a stowed configuration and are individually releasable from the endoscope E. While illustrated as outside the endoscope E, the channels C₁-Cₙ may be distributed inside the endoscope E or in a combination of internal and external endoscope positions. In use, the endoscope E of FIG. 30 would be maneuvered into a body portion or region and selectively detach external channels to provide working channel access to the body portion or region. For example, the endoscope E of FIG. 30 may be positioned as illustrated in FIGS. 29A-29D to deliver working channels in support of a surgical therapeutic and/or diagnostic procedure performed on the heart H.

FIGS. 31-39C illustrate alternative aspects and further details of the rigidizable elements that may be used in conjunction with the external working channel embodiments of the present invention described above with regard to FIGS. 28B and 28D. U.S. Pat. No. 6,800,056 is incorporated herein by reference in its entirely for all purposes.

FIG. 31 shows an isometric view of a length of the working channel 1120, in this example part of the proximal portion 1122, with a section of the working channel body 1120 removed for clarity. As seen, a representative illustration of the rigidizable element 1136 may be seen disposed within rigidizable element channel or lumen 1150 within the proximal portion 1122. Lumen 1150 may be an existing working channel, i.e., an access channel for other tools, or it may be a designated channel for rigidizable element 1136 depending upon the desired application. Rigidizable element 1136 may be inserted within rigidizable element channel 1150 through a working channel handle or proximal opening and pushed proximally or, alternatively, it may be pushed proximally or pulled distally as described in FIGS. 16-21. Although rigidizable element 36 is shown in this variation as being slidably disposed interiorly of working channel body 20, it may also be disposed exteriorly of the body 20 to slide along a rigidizable element rail or exterior channel in other variations.

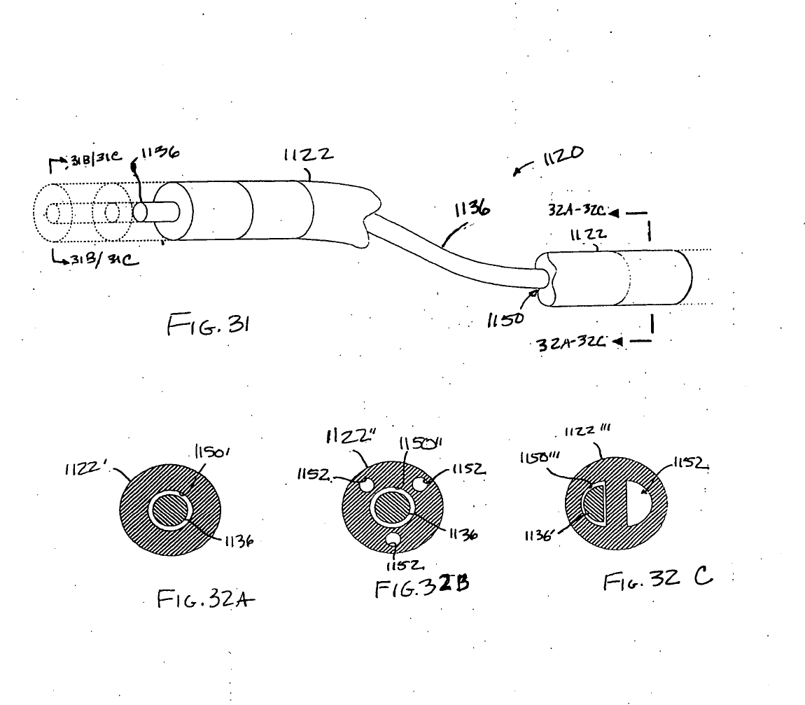

FIGS. 32A to 32C show variations on possible cross-sections 32A-32A, 32B-32B, and 32C-32C, respectively, taken from FIG. 31. FIG. 32A shows a simplified cross-section 1122′ of a rigidizable element 1136 having a circular diameter slidably disposed within proximal portion 1122. As seen, rigidizable element 1136 may be slidably positioned within channel 1150′, which may also be used as a working channel upon removal of rigidizable element 1136 during, e.g., a colonoscopy procedure, for providing access for various instruments or tools to a treatment site. FIG. 32B shows another possible variation in cross-section 1122″ where rigidizable element 1136 is positioned within channel 1150″. The variation of the proximal portion in cross-section 1122.varies. may include a number of access lumens 1152 optionally formed within the body of the device 1120. These lumens 1152 may run through the length of device 1120 and may be used for various applications, e.g., illumination fibers, laparoscopic tools, etc. Although three lumens 1152 are shown in the figure, any number of channels as practically possible may be utilized depending upon the application at hand. FIG. 32C shows another variation in cross-section 1122′″. In this variation, rigidizable element 1136′ may be formed into a semi-circular or elliptical shape to slide within a similarly shaped channel 1150′″. In this example, proximal portion 1122′″ also includes a working channel 1152′ which may be shaped accordingly to fit within the body 1122′″ along with channel 1150′″ to maintain a working channel without having to remove rigidizable element 1136′.

In any of the above examples, the working or rigidizable element channels may be integral structures within the body of working channel 1120. Having an integral structure eliminates the need for a separate lumened structure, e.g., a separate sheath, through which rigidizable element 1136 or any other tools may be inserted. Another variation utilizing multiple channels and multiple rigidizable elements will be described in further detail below. These variations are not intended to be limiting but are merely presented as possible variations. Other structures and variations thereof may be recognized by one of skill in the art and are intended to be within the scope of the claims below.

The structure of the rigidizable element may be varied according to the desired application. The following description on the rigidizable element is presented as possible variations and are not intended to be limiting in their structure. FIGS. 33A and 33B show cross-sectioned end and side views, respectively, of a guiding apparatus variation which is rigidizable by a vacuum force applied within the rigidizable element. It is preferable that the rigidizable element is selectively rigidizable, i.e., when the rigidizable element assumes a shape or curve in a flexible state, the rigidizable element may be rigidized to hold that shape or curve for a predetermined period of time. Although the working channel structure of the present invention may utilize a rigidizable element which remains in a relatively flexible shape, it is preferable to have the rigidizable element be selectively rigidizable.

Rigidizable element 1160 may be comprised of two coaxially positioned tubes, outer tube 1162 and inner tube 1164, which are separated by a gap 1166 between the two tubes. Inner tube 1164 may define an access lumen 1168 throughout the length of the tube to provide a channel for additional tools or other access devices. Both tubes 1162, 1164 are preferably flexible enough to be bent over a wide range of angles and may be made from a variety of materials such as polymers and plastics. They are also preferably flexible enough such that either the outer tube 1162, inner tube 1164, or both tubes are radially deformable. Once rigidizable element 1160 has been placed and has assumed the desirable shape or curve, a vacuum force may be applied to draw out the air within gap 1166. This vacuum force may radially deform inner tube 1164 and bring it into contact with the inner surface of outer tube 1162 if inner tube 1164 is made to be relatively more flexible than outer tube 1162. Alternatively, if outer tube 1162 is made to be relatively more flexible than inner tube 1164, outer tube 1162 may be brought into contact with the outer surface of inner tube 1164.

In another variation, tubes 1162, 1164 may both be made to be flexible such that they are drawn towards one another. In yet another variation, which may be less preferable, a positive force of air pressure or a liquid, e.g., water or saline, may be pumped into access lumen 1168. The positive pressure from the gas or liquid may force the walls of inner tube 1164 radially into contact with the inner surface of outer tube 1162. In any of these variations, contact between the two tubular surfaces will lock the tubes 1162, 1164 together by frictional force and make them less flexible. An elastomeric outer covering 1169, or similar material, may optionally be placed upon the outer surface of outer tube 1162 to provide a lubricious surface to facilitate the movement of rigidizable element 1160 within the endoscopic device. An example of a device similar to rigidizable element 1160 is discussed in further detail in U.S. Pat. No. 5,337,733, which has been incorporated herein by reference in its entirety.

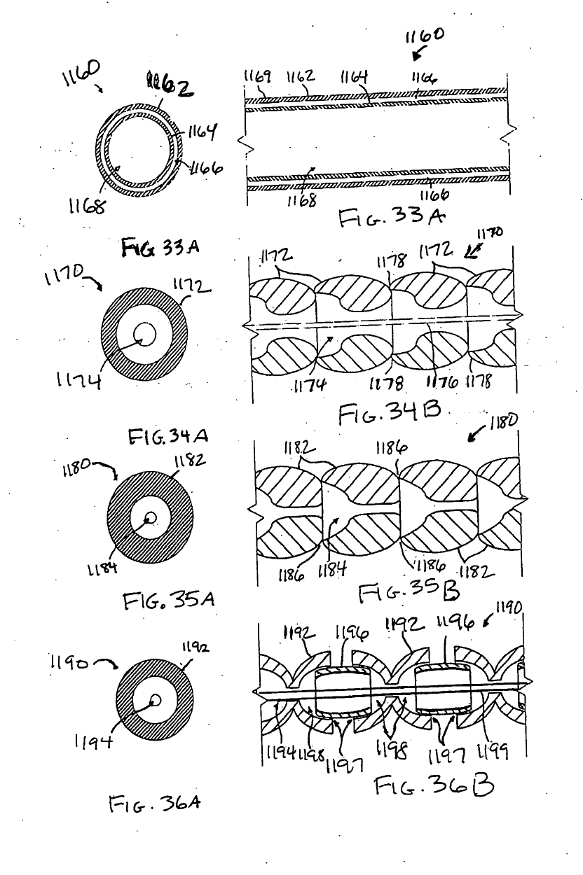

Another variation on the rigidizable element is shown in FIGS. 34A and 34B which show cross-sectioned end and side views, respectively, of a guiding apparatus variation 1170 which is rigidizable by a tensioning member 1176. Tensioned rigidizable element 1170 is shown comprised of a series of individual segments 1172 which are rotatably interlocked with one another in series. Each segment 1172 may contact an adjoining segment 1172 along a contacting lip 1178. Each segment 1172 may further define a channel therethrough which, collectively along with the other segments 1172, form a common channel 1174 throughout a majority of the length of rigidizable element 1170. Segments 1172 may be comprised of a variety of materials suitable for sustaining compression forces, e.g., stainless steel, thermoplastic polymers, plastics, etc.

Proximal and distal segments of rigidizable element 1170 may hold respective ends of tensioning member 1176, which is preferably disposed within common channel 1174 through rigidizable element 1170. Tensioning member 1176 may be connected to a tensioning housing located externally of a patient. During use when the rigidizable element is advanced distally through an working channel of the present invention, tensioning member 1176 is preferably slackened or loosened enough such that rigidizable element 1170 is flexible enough to assume a shape or curve defined by the working channel. When rigidizable element 1170 is desirably situated and has assumed a desired shape, tensioning member 1176 may be tensioned. This tightening or tensioning of member 76 will draw each segment 1172 tightly against one another along each respective contacting lip 78 such that the rigidizable element 1170 becomes rigid in assuming the desired shape. A lubricious covering, e.g., elastomers, etc., may be optionally placed over at least a majority of rigidizable element 1170 to facilitate movement of the rigidizable element 1170 relative to the endoscopic device. A similar concept and design is discussed in further detail in U.S. Pat. No. 5,624,381, which has been incorporated herein by reference in its entirety.

FIGS. 35A and 35B show cross-sectioned end and side views, respectively, of a guiding apparatus variation 1180 which is rigidizable by a vacuum force which interlocks individual segments 1182. Each segment 1182 may be adjoined with adjacent segments by interlocking ball-and-socket type joints which are preferably gasketed at the interfaces 1186 of each connection. Within each segment 1182, with the exception of the distal segment, may be defined a channel which is narrowed at one end and flared at the opposite end. Collectively when the segments 1182 are adjoined into the structure of rigidizable element 1180, each of the individual channels form a common channel 1184 which extends through at least a majority of the segments 1182 along the length of rigidizable element 1180. At the proximal end of rigidizable element 1180 a vacuum pump, which is preferably located externally of the patient, is fluidly connected to common channel 1184. In use, once rigidizable element 1180 is manipulated in its flexible state within the working channel to assume the desired shape or curve, ambient pressure may exist within common channel 1184.

When the rigid shape of rigidizable element 1180 is desired, the pump may then be used to create a negative pressure within common channel 1184 and this negative pressure draws each segment 1182 into tight contact with one another to maintain the desired shape. When the vacuum force is released, each segment 1182 would also be released and would thereby allow the rigidizable element 1180 to be in its flexible state for advancement or withdrawal. Rigidizable element 80 may further be surrounded by an elastomeric or lubricious covering to aid in the advancement or withdrawal of the rigidizable element 80 within the endoscopic device.

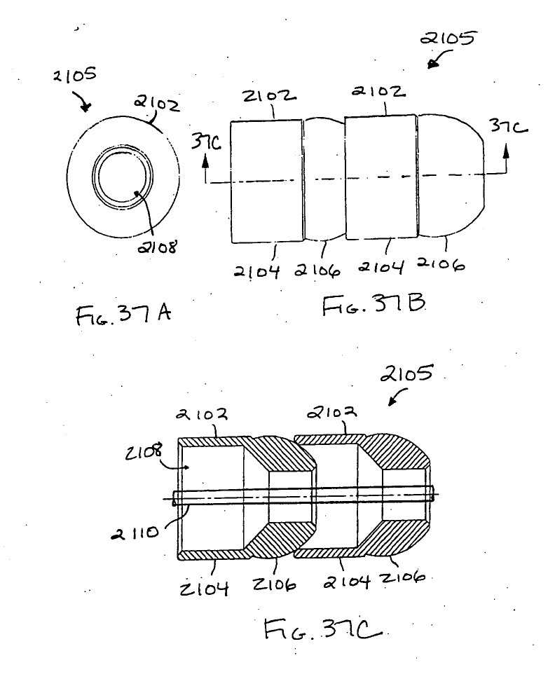

FIGS. 36A and 36B show cross-sectioned end and side views, respectively, of yet another guiding apparatus variation 1190 which is optionally rigidizable by either a vacuum force or a tensioning member which interlocks individual segments 1192. Segment 1192 may be in the form of a segmented design with two opposed cups having a common channel 1194 defined therethrough. Between each segment 1192 are ball segments 1196 which interfits along a contact rim or area 1197 within each adjacent segment 1192. Ball segments 1196 preferably contact adjacent cupped segments 96 within receiving channels 1198 defined in each cup. When manipulated in its flexible state, rigidizable element 1190 may be advanced or withdrawn or made to assume a desired shape or curve. When rigidizable element 1190 is to be placed into its rigidized shape, a vacuum force or tensioning member 1199 may be utilized in the rigidizable element 1190 in similar manners as described above. Moreover, rigidizable element 1190 may similarly be surrounded by an elastomeric or lubricious covering to aid in the advancement and withdrawal of the rigidizable element 1190.

FIGS. 37A and 37B show representative end and side views, respectively, of another guiding apparatus variation 2105. This variation 2105 comprises individual segments 2102 having a uniform sleeve section 2104 in combination with an integrated curved or hemispherical section 2106. Each segment 2102 is collinearly aligned with one another with the sleeve section 2104 receiving the curved section 106 of an adjacent segment 2102, as shown in FIG. 37C, which is the cross-section of rigidizable element 100 from FIG. 37B. The adjacent segments 2102 may rotate relative to one another over the sleeve-hemisphere interface while maintaining a common channel 2108 through the rigidizable element 2105. A tensioning member 2110 may pass through channel 2108 along the length of rigidizable element 2105 for compressing the individual segments 2102 against one another when the entire rigidizable element 2105 is rigidized.

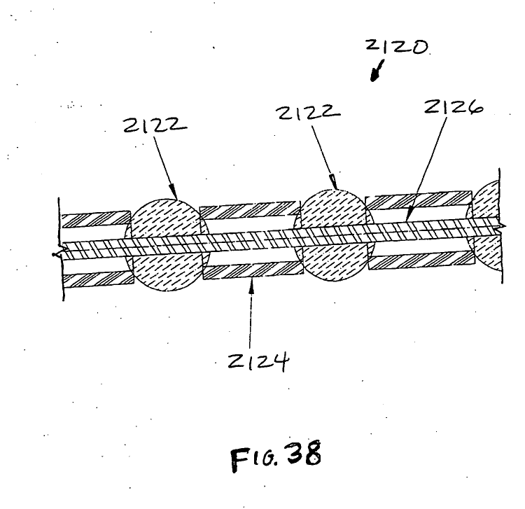

FIG. 38 shows the cross-section of another variation 2120 of the rigidizable rigidizable element apparatus. Representative segments are shown comprising spherical bead segments 2122 alternating with sleeve segments 2124. Each of the bead and sleeve segments 2122, 2124, respectively, may have a channel defined therethrough which allows for a tensioning member 126 to be run through the length of rigidizable element 2120. The alternating segments allow for the rotation of the adjacent segments while the tensioning member 2126 allows for the compression of the segments against one another when the rigidizable element 2120 is to be rigidized in much the same manner as described above.

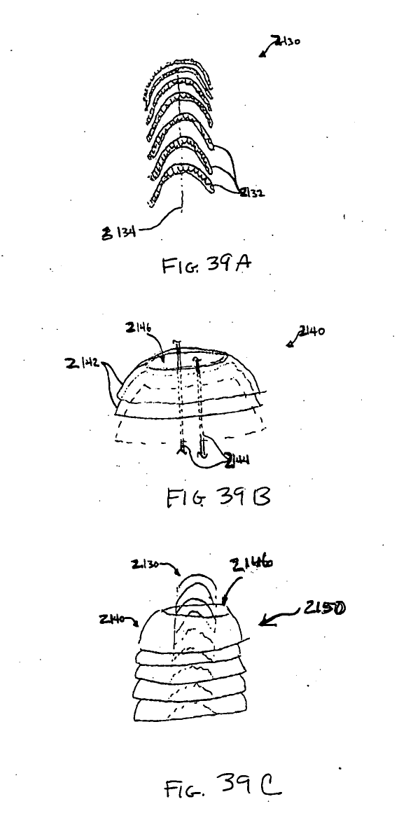

An alternative variation on the rigidizable element is illustrated in FIGS. 39A to 39C, which show a stiffening assembly having separate rigidizable coaxially positioned rigidizable elements. FIG. 39A shows a representative number of nested segments 2132 in nested stiffening assembly 2130. Each nested segment 2132 may be in a number of different configurations, e.g., ball socket joints, stacked ring-like segments, etc., with a tensioning member 2134 passing through each of the segments 2132. For use with nested assembly 2130, an annular stiffening assembly 140 may be seen in FIG. 39B. Annular assembly 2140, of which only a few representative segments are shown, are comprised in this variation of annular segments 2142 which may be stacked or aligned one atop each other. At least one tensioning member 2144, and preferably at least two, may be passed through each of the annular segments 2142. A central area 2146 is defined in each annular segment 2142 such that nested stiffening assembly 2130 may be slidingly placed within the central area 146 defined by the annular stiffening assembly 2140. FIG. 39C shows the stiffening assembly 2130 slidingly positioned within annular stiffening assembly 140 to form the coaxially aligned stiffening assembly 2150.

Still further alternative aspects of the rigidizable elements used with embodiments of the working channel of the present invention are described with regard to FIGS. 40 to 49. US Patent Application Publication 2003/0233058 filed Oct. 25, 2003 is incorporated herein by reference.

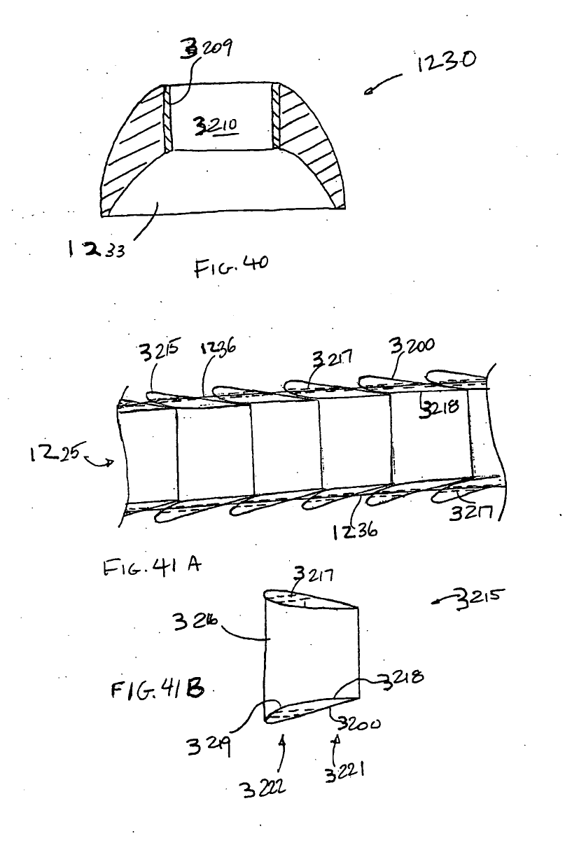

FIGS. 40, 41A, and 41B illustrate still further alternative structures to facilitate rigidizing an embodiment of a working channel of the present invention. For example, some or all of nestable rigidizable elements 1230 may incorporate hydrophilically-coated polymeric layer 3209, which may be disposed surrounding distal portion 3210 of bore 1233. A plurality of elements 1230 could be arranged along the length of a working channel as described above with regard to FIG. 28B and FIG. 28D.

Alternatively, as described in FIGS. 41A and 41B, a working channel embodiment may comprise a multiplicity of frustoconical elements 3215 that, when nested, provide a smooth inner lumen to accommodate an instrument or device therethrough without the need for a separate liner. Each frustoconical element 3215 includes central bore 3216, and at least two or more tension wire bores 3217. Central bore 3216 is defined by cylindrical distal inner surface 3218 that has a substantially constant diameter, and proximal inner surface 3219 that is continuous with distal inner surface 3218.

Proximal inner surface 3219 is slightly curved in a radially outward direction so that, when tension wires 1236 are relaxed, proximal inner surface 3219 can rotate relative to external surface 3220 of an adjacent element. External surface 3220 of each frustoconical element may be straight or contoured to conform to the shape of proximal inner surface 3219, and tapers each element so that distal end 3221 is smaller in outer diameter than proximal end 3222. When frustoconical elements 3215 are nested together, distal inner surface 3218 of each frustoconical element is disposed adjacent to the distal inner surface of an adjoining frustoconical element.

Advantageously, the present configuration provides lumen 1225 with a substantially continuous profile. This permits smooth advancement of an instrument or a device therethrough, and thereby eliminates the need to dispose a separate liner within lumen 1225. To provide a lubricious passageway to further facilitate advancement of the colonoscope, each frustoconical element optionally may incorporate an integral hydrophilic polymeric lining such as polymeric layer 209 described with respect to the preceding embodiment of FIG. 40, or a thin, flexible lining having a hydrophilic coating may be disposed through lumen 1225.

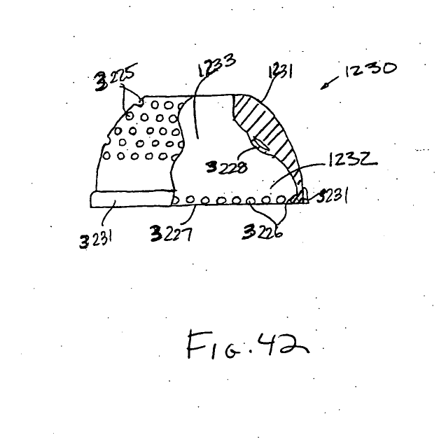

In FIG. 42, yet another alternative structure is described, in which distal surface 1231 of each nestable element is macroscopically textured to increase the friction between adjacent nestable elements 1230 when a compressive clamping load is applied. Illustratively, each element 1230 may incorporate multiplicity of divots 3225 disposed on distal surface 1231, and teeth 3226 that are disposed on proximal surface 1232 adjacent proximal edge 3227. Teeth 3226 are contoured to mate with the multiplicity of divots disposed on an adjacent element. Accordingly, tension applied to a plurality of adjacent rigidizable elements 1230 applies a clamping load to elements 1230 that causes teeth 3226 of each element to forcefully engage divots 3225 of an adjacent element. This reduces the risk of relative angular movement between adjacent nestable elements 1230 when the working channel is shape-locked, which in turn reduces the risk of undesired reconfiguration of the working channel.

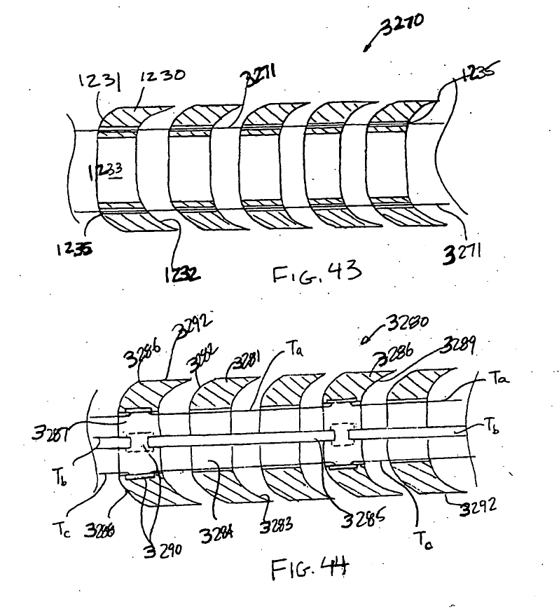

Referring now to FIGS. 43 and 44, alternative embodiments of the working channel are described. Unlike previously described embodiments, in which a mechanical mechanism is actuated to impart a clamping load to a multiplicity of nestable elements, the embodiments of FIGS. 43 and 44 use alternative tensioning mechanisms. In particular, the following embodiments comprise a multiplicity of links to which a compressive clamping load may be applied by contraction of shape memory materials.

In FIG. 43, an alternative embodiment of the working channel of the present invention is described. Working channel 3270 includes multiplicity of nestable elements 1230 identical to those described hereinabove. For purposes of illustration, nestable elements 1230 are shown spaced-apart, but it should be understood that elements 1230 are disposed so that distal surface 1231 of each element 1230 coacts with proximal surface 1232 of an adjacent element. Each of nestable elements 1230 has central bore 1233 to accommodate an instrument or a device, and preferably two or more tension wire bores 1235. When assembled as shown in FIG. 43, nestable elements 1230 are fastened with distal and proximal surfaces 1231 and 1232 disposed in a coacting fashion by a plurality of tension wires 3271 that extend through tension wire bores 1235.

In contrast to previous working channel embodiments, tension wires 3271 of the present working channel are made from a shape memory material, e.g., nickel titanium alloy, or an electroactive polymer known in the art. Tension wires 3271 are fixedly connected to the distal end of working channel 3270 at the distal ends and fixedly connected to a handle or conventional tension control system at the proximal ends. When an electric current is passed through tension wires 3271, the wires contract in length, imposing a compressive clamping load that clamps distal and proximal surfaces 1231 and 1232 of nestable elements 1230 together at the current relative orientation, thereby fixing the shape of working channel 3270. When application of electrical energy ceases, tension wires 3271 re-elongates in length to provide for relative angular movement between nestable elements 1230. This in turn renders working channel 3270 sufficiently flexible to negotiate a tortuous path through the colon, other organs or regions of the body.

To provide working channel 3270 with a fail-safe mode that reduces the risk of undesired reconfiguration of the working channel in the event of tensioning mechanism failure, diametrically disposed tension wires 3271 may be coupled in a serial circuit. Accordingly, when one wire fails, the wire disposed diametrically opposite also re-elongates to maintain a symmetrical clamping load within working channel 3270. Alternatively, all tension wires 3271 may be electrically coupled in a serial electrical circuit. Accordingly, when one of the tension wires fails, working channel 3270 returns to the flexible state.

It should be understood that a tension spring (not shown) or damper (not shown) that are familiar to those of ordinary skill may be coupled between the proximal ends of tension wires to maintain the tension wires in constant tension when the working channel is in a shape-locked state. Such constant tension reduces the risk of reconfiguration of the working channel to its flexible state if nestable elements disposed therein slightly shift relative to adjacent nestable elements.

Alternatively, as described in FIG. 44, working channel 3280 may include multiplicity of nestable elements 3281 that are similar to those of the preceding embodiments. For purposes of illustration, nestable elements 3281 are shown spaced-apart, but it should be understood that elements 3281 are disposed so that distal surface 3282 of each element 3280 coacts with proximal surface 3283 of an adjacent element. Each of nestable elements 3280 has central bore 3284 to accommodate an instrument or a device.

When assembled as shown in FIG. 44, nestable elements 3280 are fastened with distal and proximal surfaces 3282 and 3283 disposed in coacting fashion by plurality of thin tension ribbons 3285 that are fixedly connected to nestable bridge elements 3286. Tension ribbons 3285 are made from a shape memory material, e.g., nickel titanium alloy or an electroactive polymer, and may be transitioned from an equilibrium length to a contracted length when electrical current is passed therethrough.

Nestable bridge elements 3286 are disposed within working channel 3280 between a predetermined number of nestable elements 3281. Similar to nestable elements 3281, bridge elements 3286 also comprise central bore 3287 that accommodates an instrument or a device, distal surface 3288 that coacts with proximal surface 3283 of a distally adjacent nestable element, and proximal surface 3289 that coacts with distal surface 3282 of a proximally adjacent nestable element 3281. Each bridge element also incorporates plurality of conductive elements 3290 that are disposed azimuthally around central bore 3287, and that preferably couple tension ribbons 3285 occupying the same angular circumferential position within working channel 3280 in a serial electrical circuit.

When an electrical current is passed through tension ribbons 3285, the ribbons contract in length, imposing a compressive load that clamps distal and proximal surfaces of adjacent nestable elements together at the current relative orientation, thereby fixing the shape of working channel 3280. When the energy source ceases providing electricity, tension ribbons 3285 re-elongate to the equilibrium length to provide for relative angular movement between the nestable elements. This in turn renders working channel 280 sufficiently flexible to negotiate a tortuous path through the colon, another organ or region of the body.

Pursuant to another aspect of the present embodiments, tension ribbons 3285 that are disposed at diametrically opposite circumferential positions may be electrically coupled in a serial circuit. Advantageously, this configuration provides working channel 3280 with a fail-safe mode that reduces the risk of undesired reconfiguration of the working channel in the event that one of the electrical circuits established through the tension ribbons is de-energized.

For example, working channel 3280 of FIG. 44 may be provided with four sets of tension ribbons equidistantly disposed at 90 degree intervals. In the event that tension ribbons Tₐ de-energize, absent electrical communication between tension ribbons Tₐ and tension ribbons T_{c} disposed diametrically opposite thereto, working channel 3280 will spontaneously reconfigure into a new rigidized shape since the tension within the working channel no longer will be symmetrically balanced. The new shape of working channel 3280 may not replicate the selected pathway and thus may cause substantial harm to the patient.

Advantageously, the present invention may reduce the risk of undesired reconfiguration preferably by electrically coupling diametrically disposed tension ribbons in a serial circuit. When tension ribbons Tₐ are de-energized, tension ribbons T_{c} also de-energize to provide working channel 3280 with symmetrical tension, as provided by tension wires T_{b} and the tension wires disposed diametrically opposite thereto (not shown). In this manner, the working channel retains its desired rigidized shape in the event that the tensioning mechanism malfunctions. To immediately return working channel 3280 to its flexible state in the event that any of the tension ribbons are de-energized, all tension ribbons 3285 may be electrically coupled in a serial circuit.

In an alternative embodiment, tension ribbons 3285 may be electrically coupled to rigidize select regions of the working channel without rigidizing the remainder of the working channel. Illustratively, this may be accomplished by coupling longitudinally adjacent tension ribbons in a parallel circuit, and circumferentially adjacent tension ribbons in a serial circuit.

Of course, it will be evident to one of ordinary skill in the art that, while FIG. 44 depicts tension ribbons 3285 to be disposed within central bores 3284 and 3287, the tension ribbons also may be disposed adjacent external lateral surfaces 3292 of nestable elements 3281 and 3286. Alternatively, the tension ribbons may extend through tension ribbon bores (not shown) that may extend through the distal and proximal surfaces of nestable elements 3281, and be affixed to nestable bridge elements 3286. Still another alternative aspect of the use of shape memory elements in conjunction with working channel embodiments of the present invention is to transition the working channel between stowed and deployed configurations.

Referring now to FIG. 45, another alternative embodiment of a working channel is described, in which each Grecian link 3350 includes rigid first and second rims 3351 and 3352 disposed at longitudinally opposing ends of flexible body 3353. First rim 3351 comprises U-shaped arm 3354 that defines channel 3355 and opening 3356. Second rim 3352 includes retroflexed arm 3357, which when engaged to first rim 3351 of an adjacent, is disposed within channel 3355 of U-shaped arm 3354 through opening 3356 so that U-shaped arm 3354 and retroflexed arm 3357 are engaged and overlap along the longitudinal axis of the working channel.

Grecian links 3350 are disposed within compressive sleeve 3358, which includes first compressive portions 3359 and second compressive portions 3360. In compressive sleeve 3358, the second compressive portions 3360 are aligned with, and apply a clamping force to, overlapping U-shaped arm 3354 and retroflexed arm 3357 of the first and second rims. It will of course be understood that an working channel in accordance with the principles of the present invention couple alternatively be formed using Grecian links 3350 with other clamping systems known to those of ordinary skill in the art.

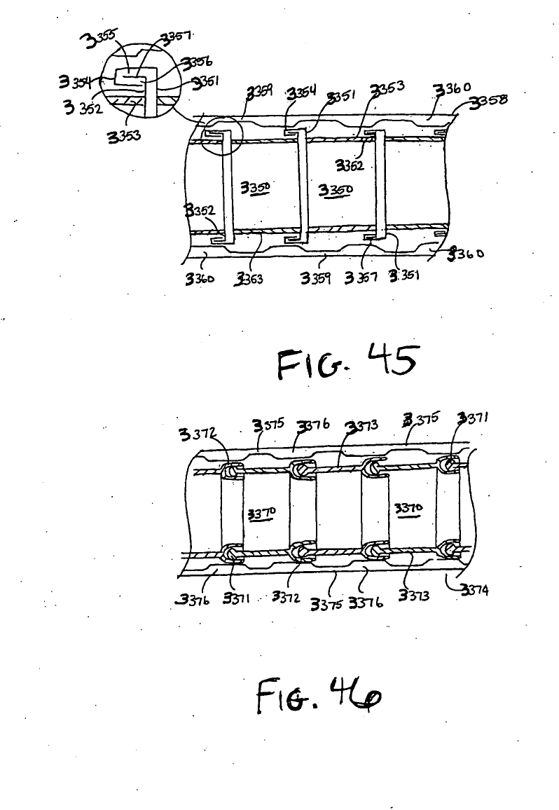

Referring now to FIG. 46, yet another alternative embodiment of an working channel suitable for use in the present invention is described. This embodiment comprises joint links 3370 that include ball 3371 and socket 3372 disposed at longitudinally opposing ends of flexible body 3373. When adjacent joint links 3370 are engaged, ball 3371 of one link is disposed within socket 3372 of an adjacent link. When the working channel is flexed, ball 3371 coacts with socket 3372 to provide articulation of the working channel.

Joint links 3370 are disposed within compressive sleeve 3374, which includes first compressive portions 3375 and second compressive portions 3376. Compressive sleeve 3374 is identical in structure and operation to that described above except that second compressive portions 3376 are aligned with, and apply a clamping force to, socket 3372 within which ball 3371 of an adjacent link is disposed. It will of course be understood that a working channel in accordance with the principles of the present invention could alternatively be formed using joint links 3370 and could employ clamping systems known to those of ordinary skill in the art.

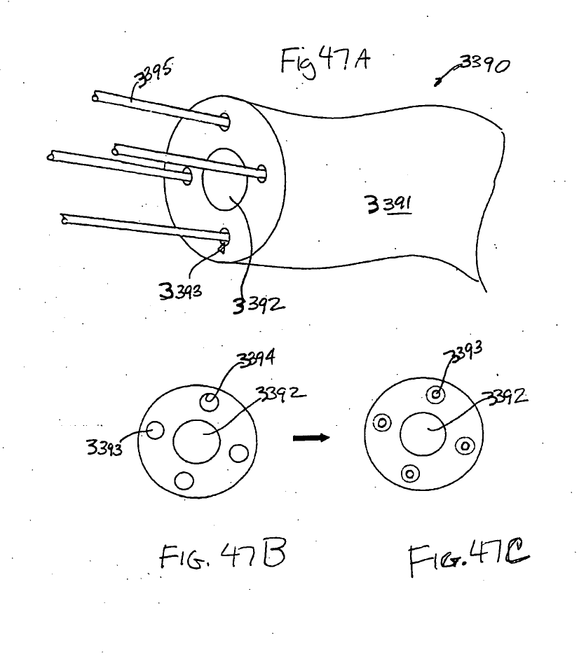

Referring now to FIGS. 47A-47C, an additional alternative embodiment of an working channel suitable for use with the present invention is described. Working channel 3390 comprises elongate body 3391 having central lumen 3392 that accommodates an instrument or a device, and wire lumens 3393 that are defined by cylindrical wire lumen surfaces 3394. Within each wire lumen 3393 is disposed wire 3395 that extends the length of the elongate body. Elongate body 3391 is made from an electroactive polymer known in the art that permits wire lumens 3393 to vary in diameter responsive to electrical energization.

In particular, when an electrical current is passed through elongate body 3391, the diameter of each wire lumen 3393 decreases so that the wire lumens clamp around respective wires 3395. Preferably, both wires 3395 and wire lumen surfaces 3394 are textured to enhance friction therebetween. This prevents further relative movement between elongate body 3391 and wires 3395, and stiffens working channel 3390. When application of the electrical current ceases, wire lumens 3393 increase in diameter to release wires 3395 so that elongate body 3391 may shift relative to wires 3395. This in turn renders working channel 3390 sufficiently flexible to negotiate a tortuous path through the colon, another organ or a body region.

With respect to FIG. 48, yet another alternative embodiment of the working channel is described. Working channel 3400 incorporates a multiplicity of variable diameter links 3401 disposed in overlapping fashion surrounding a multiplicity of rigid links 3402 that provide structural integrity to the working channel. Each link comprises a central bore that defines lumen 1225 of the working channel that is sized, when deployed, to accommodate instruments and devices. Variable diameter links 3401 preferably are manufactured from an electroactive polymer or a shape memory alloy and contract in diameter when energized. When variable diameter links 401 are electrically activated, the variable diameter links tighten about rigid links 3402 to transition working channel 3400 into a shape-locked state. When the variable diameter links are electrically deactivated, the variable diameter links sufficiently soften to return working channel 3400 back to the flexible state.

In a preferred embodiment, variable diameter links 3401 and rigid links 3402 are formed from respective strips of material that are helically wound in an overlapping fashion to form working channel 3400. Alternatively, each link may be individually formed and disposed in an overlapping fashion.

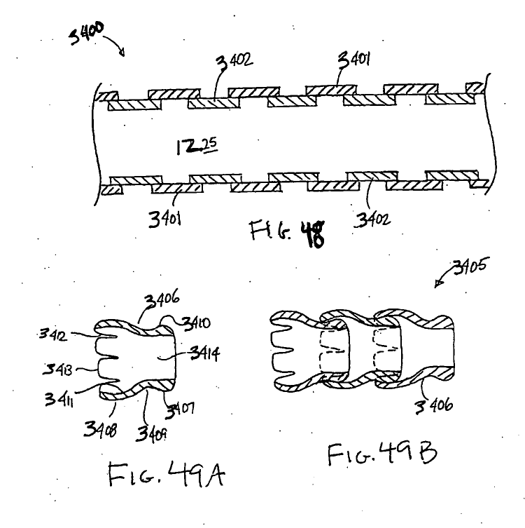

In FIGS. 49A-49B, still another alternative embodiment of an working channel suitable for use with the apparatus of the present invention is illustrated schematically. Working channel 3405 comprises a multiplicity of nestable hourglass elements 3406 that preferably are manufactured from an electroactive polymer or a shape memory alloy, and each have bulbous distal and proximal portions 3407 and 3408 connected by neck 3409. The diameter of neck 3409 is smaller than the maximum diameter of distal portion 3407, which in turn is less than the maximum diameter of proximal portion 3408. The distal portion of external surface 3410 of each hourglass element 3406 is contoured to coact with the proximal portion of internal surface 3411 of a distally adjacent hourglass element. Accordingly, when a multiplicity of hourglass elements are nested together to form working channel 3405, adjacent elements 3406 may move relative to each other when the working channel is in the flexible state.

To reduce friction between adjacent elements during relative movement therebetween, proximal portions 3408 include a plurality of slits 3412 disposed contiguous with proximal edge 3413. Slits 3412 also facilitate contraction of proximal portion 3408 of each element around distal portion 3407 of an adjacent element. Each hourglass element 3406 also has central bore 3414 that accommodates an instrument or a device.

When an electrical current is applied to the multiplicity of nestable hourglass elements 3406, proximal portion 3408 of each element contracts in diameter around distal portion 3407 of an adjacent element. The compressive clamping force thereapplied prevents relative movement between adjacent elements, thereby shape-locking the working channel. When the nestable elements are deenergized, proximal portions 3408 sufficiently relax to permit relative movement between adjacent nestable elements 3406, and thus permit working channel 3405 to negotiate tortuous curves. For purposes of illustration, it should be understood that the figures of the present application may not depict an electrolytic medium, electrodes, wiring, control systems, power supplies and other conventional components that are typically coupled to and used to controllably actuate electroactive polymers described herein.

While the illustrated embodiments described herein refer to an endoscope, it is to be appreciated that other surgical tools may be adapted to deliver external working channels of the present invention. Moreover, while described for use with controllable instruments such as endoscopes, it is to be appreciated that embodiments of the expandable working channels described herein may be used in a variety of medical, industrial and therapeutic applications.

Embodiments of the working channels of the present invention may be used not only with endoscopes but also colonoscopes, rotoscopes, cannulas, catheters, guide catheters, trocars, and in other surgical instruments used to operate in the thoracic cavity, the abdomen, the skull or within hollow body organs, or the gut. Specifically, external working channel embodiments and other improvements described herein may be modified to improve the operation and functionality of endoscopes for the examination of the esophagus, stomach, and duodenum, colonoscopes for examining the colon, angioscopes for examining blood vessels, bronchoscopes for examining bronchi, laparoscopes for examining the peritoneal cavity, arthroscopes for examining joints and joint spaces, nasopharygoscopes for examining the nasal passage and pharynx, toracoscopes for examination of the thorax and intubation scopes for examination of a person's airway.

Described here are devices, systems, and methods for navigating, maneuvering, positioning or support for delivering an instrument having an external working channel or the external working channel itself into both open and solid regions of the body. While the illustrated embodiments described to herein refer to delivery of external working channels of the present invention in conjunction with surgical, therapeutic and/or diagnostic procedures related to the colon or the heart, is to be appreciated that these are only illustrative examples.

While some specific examples are provided for a particular organ such as the colon, the invention is not so limited. It is to be appreciated that the term “region” as used herein refers to luminal structures as well as solid organs and solid tissues of the body, whether in their diseased or nondiseased state. Examples of luminal structures or lumens include, but are not limited to, blood vessels, arteriovenous malformations, aneurysms, arteriovenous fistulas, cardiac chambers, ducts such as bile ducts and mammary ducts, fallopian tubes, ureters, large and small airways, and hollow organs, e.g., stomach, small and intestines, colon and bladder. Solid organs or tissues include, but are not limited to, skin, muscle, fat, brain, liver, kidneys, spleen, and benign and malignant tumors. As such, it is to be appreciated that the external working channel embodiments of the present invention have broad applicability to numerous surgical, therapeutic and/or diagnostic procedures.

What is claimed is:

1. An apparatus, comprising:
an instrument having an elongate body;
a working channel connected externally to the elongate body and extending from a proximal position on the elongate body to a distal position on the elongate body, the working channel having a stowed configuration and a deployed configuration; and
a plurality of rigidizable elements disposed within the working channel to selectively hold the shape of the working channel when the working channel is in the deployed configuration.

2. An apparatus according to claim 1 wherein the working channel may be detached from the instrument.

3. An apparatus according to claim 1 wherein the rigidizable elements hold the shape of the working channel using mechanical force.

4. An apparatus according to claim 1 wherein the rigidizable elements hold the shape of the working channel using a shape memory alloy element.

5. An apparatus according to claim 1 wherein the rigidizable elements hold the shape of the working channel using an electroactive polymer element.

6. An apparatus according to claim 1 wherein the plurality of rigidizable elements disposed within the working channel comprises nested rigidizable elements.

7. An apparatus according to claim 1 wherein the instrument is adapted to provide more than one working channel.

8. An apparatus according to claim 7 wherein each working channel of the more than one working channel may be independently released from the instrument.

9. A method of providing a working channel within the body, comprising:
Positioning an instrument within the body,
Providing along the instrument an external working channel having a lumen, the lumen extending along the working channel and outside of the instrument;
Positioning the external working channel into a deployed configuration; and
Holding the shape of the external working channel in the deployed configuration.

10. The method according to claim 9 further comprising releasing the external working channel from the instrument so that the instrument may move axially with respect to the deployed external working channel.

11. The method according to claim 9 further comprising holding the shape of the external working channel in the deployed configuration using mechanical force produced by rigidizable elements within the external working channel.

12. The method according to claim 9 further comprising holding the shape of the external working channel in the deployed configuration using a shape memory alloy element.

13. The method according to claim 9 further comprising holding the shape of the external working channel in the deployed configuration using a electroactive polymer element.

14. The method according to claim 9 wherein the working channel is used to perform a screening, therapy or diagnostic procedure within the body.

15. The method according to claim 9 further comprising
Adjusting the position of the instrument within the body;
Providing along the instrument another external working channel having a lumen, the lumen extending along the working channel and outside of the instrument;
Positioning the another external working channel into a deployed configuration; and
Holding the shape of the another external working channel in the deployed configuration.

16. The method according to claim 14 wherein the working channel, the another working channel and the instrument are used to perform a screening, therapy or diagnostic procedure within the body.

17. A method of providing access within the body, comprising:
Positioning an instrument within the body;
Providing along the instrument a first external working channel having a lumen, the lumen extending along the working channel and outside of the instrument;
Positioning the first external working channel into a deployed configuration in a first position within the body;
Holding the shape of the first external working channel in the deployed configuration using rigidizable elements within the first external working channel;
Moving the instrument to a second position within the body;
Providing along the instrument a second external working channel having a lumen, the lumen extending along the working channel and outside of the instrument;
Positioning the second external working channel into a deployed configuration in the second position within the body; and
Holding the shape of the second external working channel in the deployed configuration using rigidizable elements within the second external working channel.

18. The method according to claim 17 further comprising performing a screening, a therapy or a diagnostic procedure within the body at either the first position or the second position.

19. The method according to claim 18 wherein the first position and the second position are adjacent the heart.

20. The method according to claim 18 wherein the first position and the second position are within the gut.

21. The method according to claim 18 wherein the screening, therapy or diagnostic procedure within the body relates to the heart.

22. The method according to claim 18 wherein the screening, therapy or diagnostic procedure within the body relates to the gut.
